(19) United States
(12) Patent Application Publication
(10) Pub. No.: US 2005/0119491 A1
(43) Pub. Date: Jun. 2, 2005

(54) 2, 7-Substituted carbazoles and oligomers, polymers and co-polymers thereof

(76) Inventors: Ahmed Iraqi, Sheffield (GB); Isao Wataru, Sheffield (GB)

Correspondence Address: FISH & RICHARDSON PC, 225 FRANKLIN ST, BOSTON, MA, 02110, US

(21) Appl. No.: 10/498,716

(22) PCT Filed: Dec. 11, 2002
(86) PCT No.: PCT/GB02/05606

(30) Foreign Application Priority Data
Dec. 12, 2001 (GB) 0129634.6

(51) Int. Cl. C08G 73/06; C07D 209/56

(52) U.S. Cl. 548/444; 528/423

(57) ABSTRACT

A carbazole of formula I wherein R₁ and R₂ are each independently halo, B(OH)₂, B(OR₇)₂, Sn(R₆), C₁₋₂₀ hydrocarbyl, or C₁₋₂₀ hydrocarbyl comprising one or more hetero atoms, R₃ is H, halo, C₁₋₂₀ hydrocarbyl, C₁₋₂₀ hydrocarbyl comprising one or more heteroatoms, or cyano, R₄ and R₅ are each independently H, halo, C₁₋₂₀ hydrocarbyl, Cₗ₋₂₀ hydrocarbyl comprising one or more heteroatoms, or cyano, and R₇ and R₈ are each independently C₁₋₂₀ hydrocarbyl, provided that R₄ and R₅ are not both H when R₃ is n-octyl, and conjugated oligomers, polymers and co-polymers thereof. Embodiments of the oligomers, polymers and co-polymers can, for example, be formed into films which can be incorporated into electronic devices.

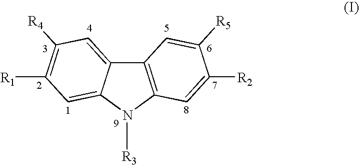

This invention relates to 2,7-disubstituted carbazoles which are substituted at the 9-position, and methods for the preparation of such 9-substituted 2,7-disubstituted carbazoles. The invention further relates to oligomers, polymers and co-polymers of such carbazoles, to films and coatings prepared from such carbazoles, oligomers, polymers and co-polymers, processes for preparing such films and coatings, and electroluminescent and other electronic devices comprising one or more layers of polymer films, at least one of which layers is derived from the oligomers, polymers and co-polymers of the invention. The invention still further relates to processes for the post-modification of such carbazole oligomers, polymers and co-polymers.

The preparation of 2,7-dibromocarbazole has been reported by Yamato et al (Journal of Organic Chemistry, Volume 56, pages 6248-50, 1991) and Patrick et al (Eur. J. MED. Chem Volume 32 pages 781-793, 1997). 2,7-linked carbazole polymers substituted by octyl groups at the 9-position have also been reported by Geissler et al (Polym. Adv. Technol. Volume 8 pages 87-92, 1997). These polymers were prepared by the Kumada procedure wherein the 2,7-dibromo-carbazole monomers were treated with one mole equivalent of magnesium in the presence of palladium dichloride. The structures are represented by Geissler et al as poly (9-alkylcarbazole-2,7-diyls) with degrees of polymerisation not exceeding 4 as measured by gel permeation chromatography.

Leclerc et al (Macromolecules, volume 34, pages 4680-4682, 2001) have also reported the preparation of poly CONFIRMATION COPY (9)-(2-ethylhexyl)carbazole-2,7-diyls) and poly(9)-(octyl)carbazole-2,7-diyls (on treatment of the respective 2,7-dichloro- or 2,7-diiodo-9-alkyl-carbazole derivatives with nickel dichloride in the presence of 2,2,-bipyridine, triphenylphosphine and zinc. The resulting homopolymers were sparingly soluble in common organic solvents. The same group have also reported (Macromolecules, volume 34, pages 4680-4682, 2001) the preparation of alternating carbazole co-polymers comprising 9,9′-dialkyl-fluorene or 2,2′-bithiophene using the above 2,7-dichloro- or 2,7-diiodo-9-alkyl-carbazole monomers in Suzuki and Stille type coupling reactions.

The present invention provides novel 2,7-disubstituted carbazoles and methods for preparing oligomers, polymers and copolymers having improved properties from such 2,7-disubstituted carbazoles in high yield. Carbazole polymers according to the invention can exhibit low polydispersity and high glass transition temperatures.

In a first aspect, the invention provides a carbazole of formula: 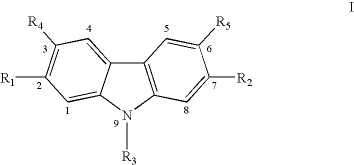
wherein R₁, and R₂ are each independently halo, B(OH)₂, B (OR₇)₂, Sn (R₈)₃, C₁₋₂₀ hydrocarbyl, or C₁₋₂₀ hydrocarbyl comprising one or more hetero atoms,
R₃ is H, halo, C₁₋₂₀ hydrocarbyl, C₁₋₂₀ hydrocarbyl comprising one or more heteroatoms, or cyano,
R₄ and R₅ are each independently H, halo, C₁₋₂₀ hydrocarbyl, C₁₋₂₀ hydrocarbyl comprising one or more heteroatoms, or cyano, and
R₇ and R₈ are each independently C₁₋₂₀ hydrocarbyl, provided that R₄ and 5 are not both H when R₃ is n-octyl.

Preferred compounds of formula I are those wherein R₄ and R₅ are not both H.

In a second aspect, the invention provides a conjugated oligomer or polymer comprising at least 1.0% of the repeating unit: 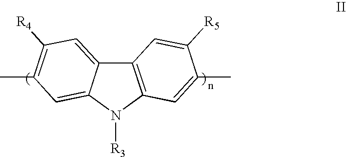
wherein R₃ is HI halo, C-₁₋₂₀ hydrocarbyl, C₁₋₂₀ hydrocarbyl comprising one or more heteroatoms, or cyano,
R₄ and R₅ are each independently H, halo, C₁₋₂₀ hydrocarbyl, C₁₋₂₀ hydrocarbyl comprising one or more hetero atoms, or cyano,
and wherein the polymer has a degree of polymerisation n greater than 4 as measured by gel permeation chromotography.

Preferred oligomers and polymers according to the second aspect of the invention are those wherein at least one of R₄ and R₅ is not H. Especially preferred are 9-substituted carbazole oligomers and polymers, which are terminated at the terminal 2- and 7′-positions with hydrogen or a halogen atom.

In a third aspect, the invention provides a co-polymer of the formula: 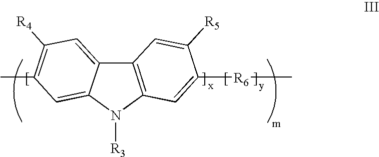
wherein R₃, R₄ and R₅ represent substituents as hereinbefore defined,
R₆ is an aryl or heteroaryl repeating unit,
0.1<x<1, 0.1<y<0.9, x+y=1, and
m is an integer greater than 1.

In this specification “hydrocarbyl” means any organic moiety containing only hydrogen and carbon unless specified otherwise and may include substituted and unsubstituted aliphatic, cycloaliphatic and aromatic, moieties and moieties containing two or more of aliphatic cycloaliphatic and aromatic moieties.

In a fourth aspect the invention provides a method for the production of a compound of formula I where R₃ is H, which comprises contacting a 4,5,4′, 5′-tetrasubstituted-biphenyl-2,2′-diamine derivative of formula: 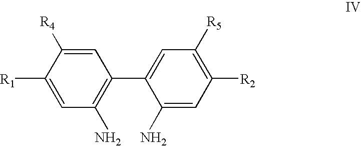
Where R₁, R₂, R₄, and R₅ represent substituents as hereinbefore defined, with an arylsulphonic acid in an organic solvent at an elevated reaction temperature.

The elevated reaction temperature is preferably greater than 140° C., more preferably greater than 180° C. and most preferably between 180° C. and 250° C. The aryl group of the arylsulphonic acid preferably comprises a mono-, di- or tri-substituted benzene ring where the one or more substituent groups is/are preferably a C₁₋₂₀ hydrocarbyl group, or a C₁₋₂₀ hydrocarbyl group comprising one or more S, N, O, P or Si atoms.

In a fifth aspect the invention provides a method for the production of a polymer of formula XI or a copolymer of formula III by reacting a compound of formula I in a metal-catalysed coupling reaction.

In a sixth aspect the present invention provides a film comprising at least 0.1 weight % of at least one oligomer, polymer and/or co-polymer of the invention.

In a seventh aspect the invention provides an electroluminescent device, or other electronic device, comprising one or more layers of polymer films, at least one of which is derived from the oligomers, polymers and copolymers of the invention.

In formulae I and I₁, R₄ and R₅ are preferably independently in each occurrence hydrogen, C₁₋₂₀ hydrocarbyl, C₁₋₂₀ hydrocarbyloxy, C₁₋₂₀ thiohydrocarbyloxy, or cyano. More preferably R₄ and R₅ are independently in each occurrence hydrogen, C₁₋₂₀ alkyl, C₆₋₁₀ aryl or alkyl-substituted aryl, C₆₋₁₀ aryloxy or alkyl-substituted aryloxy, C₁₋₁₂ alkoxy/thioalkoxy, and cyano. Even more preferably R₄ and R₅ are independently in each occurrence hydrogen, C₁₋₁₀ alkyl, phenyl, and cyano.

R₃ can be hydrogen, C₁₋₂₀ hydrocarbyl, optionally substituted with one or more of C₁₋₂₀ alkoxy/aryloxy, thioalkoxy/thioaryloxy, secondary/tertiary amines, hydroxy, carboxylic/sulphonic acids, cyano, and esters; or C₆₋₂₀ aryl, optionally substituted with C₁₋₂₀ alkoxy/aryloxy, thioalkoxy/thioaryloxy, secondary/tertiary amines, hydroxy, carboxylic/sulphonic acids, cyano, and esters. R₃ may be a cyclic structure which may contain one or more heteroatoms such as phosphorus, sulphur, oxygen and nitrogen. Preferably, R₃ is hydrogen, C₁₋₁₂ alkyl, optionally substituted with one or more C₁₋₁₂ alkoxy/aryloxy, thioalkoxy/thioaryloxy, secondary/tertiary amines, hydroxy, carboxylic/sulphonic acids, cyano and esters; or C₆₋₂₀ aryl optionally substituted with C₁₋₁₂ alkoxy/aryloxy, thioalkoxy/thioaryloxy, secondary/tertiary amines, hydroxy, carboxylic/sulphonic acids, cyano, and esters. Most preferably R₃ is hydrogen, C₁₋₈ alkyl, optionally substituted with C₁₋₁₀ alkoxy/aryloxy, thioalkoxy/thioaryloxy, secondary/tertiary amines, hydroxy, carboxylic/sulphonic acids, cyano, and esters; or C₆₋₁₂ aryl, optionally substituted with C₁₋₁₀ alkoxy/aryloxy, thioalkoxy/thioaryloxy, secondary/tertiary amines, hydroxy, carboxylic/sulphonic acids, cyano, and esters.

Preferred compounds of formula I are: 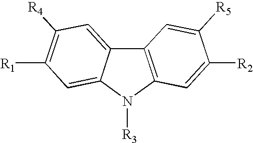
wherein R₁ and R₂ are each independently Cl, Br, I, B(OH)₂, B(OR₇)₂, Sn(R₈)₃, C₁₋₂₀ hydrocarbyl, or C₁₋₂₀ hydrocarbyl comprising one or more S, N, O, P or Si atoms,
R₃ is H, C₁₋₂₀ hydrocarbyl, or C₁₋₂₀ hydrocarbyl comprising one or more S, N, O, P or Si atoms,
R₄ and R₅ are each independently H, C₁₋₂₀ hydrocarbyl, or C₁₋₂₀ hydrocarbyl comprising one or more S, N, O, P or Si atoms, and
R₇ and R₈ are each independently C₁₋₂₀ hydrocarbyl, Provided that R₄ and R₅ are not both H when R₃ is n-octyl.

Examples of preferred compounds of formula I according to the invention where R₁ and R₂ are halo or hydrocarbyl, R₃ is H and 4 and R₅ are hydrogen atoms or hydrocarbyl groups include 2,7-dimethyl-carbazole, 2,7-dichloro-carbazole, 2,7-dibromo-carbazole, 2,7-diiodo-carbazole, and 2,7-dibromo-3,6-dimethyl-carbazole.

Examples of preferred compounds of formula I according to the invention where R₁ and R₂ are halide, where R₃ is a hydrocarbyl group and R₄ and R₅ are independently hydrogen atoms or hydrocarbyl groups include 2,7-dibromo-9-(2-ethylhexyl)-carbazole, 2,7-dibromo-g-dodecyl-carbazole, 2,7-dibromo-9-hexadecyl-carbazole, 2,7-dibromo-9-(2-hexyldecyl)-carbazole, 2,7-dibromo-3,6-dimethyl-9-(2-hexyldecyl)-carbazole, 2,7-dichloro-9-dodecyl-carbazole, and 2,7-dichloro-9-(2-hexyldecyl)-carbazole.

Examples of preferred compounds of formula I according to the invention where R₁ and R₂ are boronic acid ester groups or boronic acid groups, R₃ is a hydrocarbyl group and R₄ and R₅ are hydrogen atoms or hydrocarbyl groups include 2,7-bis(boronic acid)-9-hexadecyl-carbazole, 2,7-bis(4,4,5,5-tetramethyl-1,3,2-dioxaborolan-2-yl)-9-hexadecyl-carbazole, 2,7-bis (boronic acid)-3,6-dimethyl-9-hexadecyl-carbazole, 2,7-bis(boronic acid)-9-(2-hexyldecyl)-carbazole, 2,7-bis(4,4,5,5-tetramethyl-1,3,2-dioxaborolan-2-yl)-9-(2-hexyldecyl)-carbazole, and 2,7-bis(boronic acid)-3,6-dimethyl-9-(2-hexyldecyl)-carbazole.

Examples of preferred compounds of formula I according to the invention where R₁ and R₂ are trialkylstannyl groups, R₃ is a hydrocarbyl group and R₄ and R₅ are hydrogen atoms or hydrocarbyl groups include 2,7-bis(tri-(n-butyl) stannyl)-9-(2-hexyldecyl)-carbazole and 2,7-bis(tri-(n-methyl)stannyl)-3,6-dimethyl-9-(2-hexyldecyl)-carbazole.

Preferred compounds of formula II are: 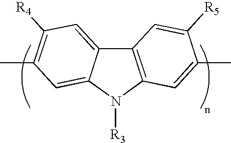
wherein R₃ is C₁₋₂₀ hydrocarbyl, or C₁₋₂₀ hydrocarbyl comprising one or more S, N, O, P or Si atoms,
R₄ and R₅ are each independently H, Cl, Br, I, C₁₋₂₀ hydrocarbyl, or C₁₋₂₀ hydrocarbyl comprising one or more S, N, O, P or Si atoms, and
n is greater than 4.

Examples of preferred compounds of formula II according to the invention where R₃ is a hydrocarbyl group and R₄ and R₅ are each independently hydrogen atoms or hydrocarbyl groups include poly(9-dodecylcarbazole)-2,7-diyl, poly(9-hexadecylcarbazole)-2,7-diyl, poly(9-(2-hexyldecyl) carbazole)-2,7-diyl, poly(3,6-dibromo-9-(2-hexyldecyl)-carbazole)-2,7-diyl and poly(3,6-dimethyl-9-(2-hexyldecyl)-carbazole)-2,7-diyl.

Preferred compounds of formula III are: 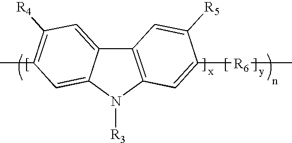
wherein R₃ is C₁₋₂₀ hydrocarbyl, or C₁₋₂₀ hydrocarbyl comprising one or more S, N, O, P or Si atoms,
R₄ and R₅ are each independently H, Cl, Br, I, C₁₋₂₀ hydrocarbyl or C₁₋₂₀ hydrocarbyl comprising one or more S,
N, O, P or Si atoms
R₆ is an aryl or heteroaryl repeating unit,
0.1<x<0.9, 0.1<y<0.9, x+y 1 and
m is an integer greater than 1.

Examples of preferred compounds of formula III according to the invention where 3 is a hydrocarbyl group, R₁ and R₅ are hydrogen atoms or hydrocarbyl groups and R₆ is an aryl or heteroaryl repeat unit include poly{(2,2′-bithiophene)-5,5′-diyl-alt-co-(9-(2-hexyldecyl)-carbazole)-2,7-diyl)}, poly{(2,5-bis(decyloxy)-benzene-1,4-diyl)-alt-co-(9-(2-hexyldecyl)-carbazole)-2,7-diyl)}, poly{(9-(2-hexyldecyl)-carbazole)-2,7-diyl)-alt-co-(naphthalene-1,4-diyl)}, a statistical copolymer comprising 85% of 2,7-linked-(9-(2-hexyldecyl)-carbazole) and 15% of 1,4-linked-naphthalene, a statistical copolymer comprising 85% of 2,7-linked-(9-(2-hexyldecyl)-3,6-dimethyl-carbazole) and 15% of 1,4-linked-naphthalene, a statistical copolymer comprising 85% of 2,7-linked-(9-(2-hexyldecyl)-3,6-dimethyl-carbazole) and 15% of 1,4-linked-(2,5-bis-(n-hexyl)-benzene), a statistical copolymer comprising 85% of 2,7-linked-(9-(2-hexyldecyl)-3,6-dimethyl-carbazole) and 15% of 3,8-linked-[1,10]phenanthroline and a statistical copolymer comprising 80% of 2,7-linked-(9-(2-hexyldecyl)-3,6-dimethyl-carbazole) and 20% of 4,4′-linked-(2,5-diphenyl-[1, 3, 4] oxadiazole).

The compounds of formula I where R₃ is H may be made by contacting a 4,5,4′,5′-tetrasubstituted-biphenyl-2,2′-diamine derivative with an arylsulphonic acid such as dodecylbenzenesulfonic acid in an organic solvent with a high boiling point such as 4-tert-butyl-o-xylene or 5-tert-butyl-m-xylene at reflux temperatures. This new synthetic method provides 2,7-dihalo-carbazoles, 2,7-dialkyl-carbazoles and 2,7-dihalo-3,6-dialkyl-carbazoles such as 2,7-dimethyl-carbazole, 2,7-dichloro-carbazole, 2,7-dibromo-carbazole, 2,7-dibromo-carbazole and 2,7-dibromo-3,6-dimethyl-carbazole in high yields (85 to 96% yields).

The 2,7-dihalo-carbazole derivatives are then further functionalised to afford a series of other novel compounds of formula I where R₃ is a C₁₋₂₀ hydrocarbyl group, or a C₁₋₂₀ hydrocarbyl group comprising one or more S, N, O, P or Si atoms. For example, alkylation of the nitrogen atom of these 2,7-dihalo-carbazole derivatives in the presence of tetra-n-butylammonium hydrogensulfide and NaOH in acetone at reflux affords a series of 9-functionalised carbazole derivatives with hydrocarbyl groups such as 2,7-dibromo-9-(2-hexyldecyl)-carbazole, 2,7-dibromo-3,6-dimethyl-9-(2-hexyldecyl)-carbazole, 2,7-dichlorocarbazole-9-dodecyl-carbazole and 2,7-dichloro-9-(2-hexyldecyl)-carbazole in high yields (86 to 94% yields).

Compounds of formula I where R₁ and R₂ are boronic acid ester groups or boronic acid groups and where R₃ is a C₁₋₂₀ hydrocarbyl group or a C₁₋₂₀ hydrocarbyl group containing one or more S, N, O, P or Si atoms can be made from the corresponding 2,7-dihalo-carbazole derivatives. The boronic acid ester derivatives are obtained from the corresponding 2,7-dihalo-monomers upon metal halogen exchange reactions at low temperature and further reaction with tri-alkoxy-borane derivatives. In situ hydrolysis of the boronic acid ester derivatives affords the boronic acid derivatives. Examples of preferred compounds prepared following this procedure include 2,7-bis(boronic acid)-9-hexadecyl-carbazole, 2,7-bis(4,4,5,5-tetramethyl-1,3,2-dioxaborolan-2-yl)-9-hexadecyl-carbazole, 2,7-bis(boronic acid)-3,6-dimethyl-9-hexadecyl-carbazole, 2,7-bis(boronic acid)-9-(2-hexyldecyl)-carbazole, 2,7-bis(4,4,5,5-tetramethyl-1,3,2 dioxaborolan-2-yl)-9-(2-hexyldecyl)-carbazole and 2,7-bis(boronic acid)-3,6-dimethyl-9-(2-hexyldecyl)-carbazole.

Compounds of formula I where R₁ and R₂ are trialkylstannyl groups and where R₃ is a C₁₋₂₀ hydrocarbyl group or C₁₋₂₀ hydrocarbyl group comprising one or more S, N, O, P or Si atoms can be made from the corresponding 2,7-dihalo-carbazole derivatives. The trialkylstannyl derivatives are obtained from the corresponding 2,7-dihalo-monomers upon metal halogen exchange reactions at low temperature and further reaction with tri-alkyltin chloride derivatives. The compounds are preferably purified on basic chromatographic beds. Examples of preferred compounds prepared following this procedure include 2,7-bis(tri-(n-butyl)stannyl)-9-(2-hexyldecyl)-carbazole and 2,7-bis (tri-(n-methyl) stannyl)-3,6-dimethyl-9-(2-hexyldecyl)-carbazole.

The compounds of the first aspect of the invention are useful in the preparation of the polymers and copolymers of the second and third aspects of the invention.

The polymers and co-polymers of the present invention may be prepared by a variety of polycondensation processes. Particularly effective are those processes involving coupling of aromatic/vinylic/acetylenic monomers catalyzed by transition metals such as nickel, and especially palladium.

The polycondensation is preferably carried out in an organic solvent, for example, tetrahydrofuran and preferably in a sealed vessel.

Other methods which can be utilised to form the polymers and copolymers of the invention from compounds of formula I are taught by Miyaura and Suzuki (Chemical Reviews, volume 95, 1995, pages 2457-2483). This reaction has been adapted with improvement for the production of higher molecular weight polymers as described in U.S. Pat. No. 5,777,070. The entire disclosures of these documents are incorporated herein by reference for all purposes.

The oligomers, polymers and copolymers of the invention when made according to the method of the invention do not contain a significant amount of misformed polynuclear structures or bonding through positions other than the 2- and 7′-positions, and they can be converted into films that are useful as light-emitting or carrier transport layers in light-emitting diodes. The polymers have good solubility characteristics and relatively high glass transition temperatures, which facilitate their fabrication into coatings and films that are relatively, thermally stable, and relatively free of defects. If the polymers contain end groups which are capable of being cross linked, the cross linking of such groups after the films or coating is formed increases the solvent resistance thereof, which is beneficial in applications wherein one or more solvent-based layers of material are deposited thereon.

The co-polymers of the third aspect of the invention comprise at least 10%, based on residual monomeric units (RMU), of 9-substituted carbazole moieties represented by formula III. A residual monomeric unit is the portion of the monomer that is incorporated into the polymer backbone. Preferably R₆ in formula III is a C₄₋₂₀ unsaturated ring structure containing optionally one or more heteroatoms of S, I, or O.

Preferably the copolymers of the invention comprise at least 20% by weight of RMUs of formula III, and more preferably at least 25% by weight, most preferably at least 50% by weight.

The polymers and co-polymers of the invention are characterised by their excellent solubility (>1 g/L) in common organic solvents, ability to form pin-hole free films and weight-average molecular weights of at least 3000 gram/mole relative to polystyrene standard, preferably at least 6000 gram/mole, more preferably 4% least 10000 gram/mole and most preferably at least 20000 gram/mole. They are further characterised by a polydispersity of less than 10, preferably less than 5, most preferably less than 3.

The present invention also envisages the modification of the abovementioned polymers and co-polymers by the introduction of further and/or alternative substituent groups by methods known in the art. Such methods include, for example, electrophilic substitution of preformed polymers at the 3- or the 3,6-positions on carbazole repeat units upon reaction with N-bromosuccinimide (NBS) or N-chlorosuccinimide (NCS). Poly(3,6-dibromo-9-(2-hexyldecyl)-carbazole)-2,7-diyl, for example, was prepared by reacting poly(9-(2-hexyldecyl)carbazole)-2,7-diyl with 2.4 equivalents of NBS in chloroform. NMR analysis reveals the total disappearance of hydrogen signals at the 3,6-positions of the carbazole repeat units. The new poly (3,6-dibromo-9-(2-hexyldecyl)-carbazole)-2,7-diyl obtained can serve itself as a precursor polymer for further functionalisation upon Grignard cross-coupling reactions. For example, poly(3,6-dimethyl-9-(2-hexyldecyl)-carbazole)-2,7-diyl can be obtained upon reaction of poly(3,6-dibromo-9-(2-hexyldecyl)-carbazole)-2,7-diyl with an excess of methyl magnesium iodide in the presence of 1,3-bis-(diphenylphosphino)-propane-dichloronickel (II). NMR analysis of this polymer gives identical spectral features to those of poly(3,6-dimethyl-9-(2-hexyldecyl)-carbazole)-2,7-diyl made by direct polymerisation of 2,7-dibromo-3,6-dimethyl-9-(2-hexyldecyl)-carbazole).

Embodiments of polymers and co-polymers of the invention exhibit photoluminescent emission in the range of 350 nm to 1000 nm and absorption from 200 nm to 600 nm. The polymers and copolymers of this invention may be useful inter alia as the active components in electronic devices including light emitting diodes., photocells, photoconductors and field effect transistors.

The copolymers of the invention comprise at least 10% RMU of structure III and preferably at least 1% of two or more RMUs possessing hole transporting property. Hole transporting property is imparted to a polymer by electron-rich RMUs. Examples include those derived from stilbenes or 1,4-dienes without electron-withdrawing substituents, tertiary amines, N,N,N′,N′-tetraaryl-1,4-diaminobenzene, N,N,N′,Nl-tetraarylbenzidine, diarylsilanes, and thiophenes/furans/pyrroles without electron-withdrawing substituents. These hole transporting RMUs may bear a variety of substituents so long as their presence does not significantly affect hole transporting properties adversely. Preferred substituents are C₁₋₂₀ alkyls, C₆₋₂₀ aryls and alkylaryls optionally substituted with C₁₋₆ alkoxys and C₆₋₁₂ aryloxys. Particularly effective are RMUs derived from tertiary aromatic amines, N,N,N′,N′-tetraaryl-1,4-diaminobenzene N,N,N′, N′-tetraaryl benzadine, thiophene and bithiophene.

Preferably the co-polymers comprise at least 15% of RMUs of structure III, and at least 10% of two or more hole transporting RMUs. Most preferably the co-polymers comprise at least 20% of RMUs of structure III and at least 20% of two or more RMUs possessing hole transporting property. The hole transporting RMUs in the co-polymers of the invention need not necessarily all belong to the same chemical type. A co-polymer of the invention may, for example, contain RMUs of the silanyl type, RMUs of the thiophene type and RMUs of the tertiary amine type.

In a further embodiment, the copolymers of the invention comprise at least 10 of RMUs of structure III and at least 1% of two or more RMUs possessing electron transporting property. Electron transporting property is imparted to polymers by electron-deficient RMUs. Examples include RMUs comprising electron withdrawing groups such as F, cyano, sulphonyl, carbonyl, nitro, carboxy; moieties containing imine linkages, and condensed polycyclic aromatics. Condensed polycyclic aromatics include acenaphthene, phenanthrene, anthracene, fluoranthene, pyrene, perylene, rubrene, chrysene, and corene. Five-membered heterocycles comprising imine linkages include oxazoles/isoxazoles, N-substituted-imidazoles/pyrazoles, thiazole/isothiazole, oxadiazoles, and N-substituted-triazoles. Six-membered heterocycles comprising imine linkages include pyridines, pyridazines, pyrimidines, pyrazines, triazines and tetrazenes. Benzo-fused heterocycles containg imine linkages include benzoxazoles, benzothiazole, benzimidazoles, quinoline, isoquinolines, cinnolines, quinazolines, quinoxalines, phthalazines, benzothiadiazoles, benzotriazines, phenazines, phenanthridines, and acridines. More complex RMUs include 1,4-tetrafluorophenylene, 1,4′-octafluorobiphenylene, 1,4-cyanophenylene, 1,4-dicyanophenylene, and

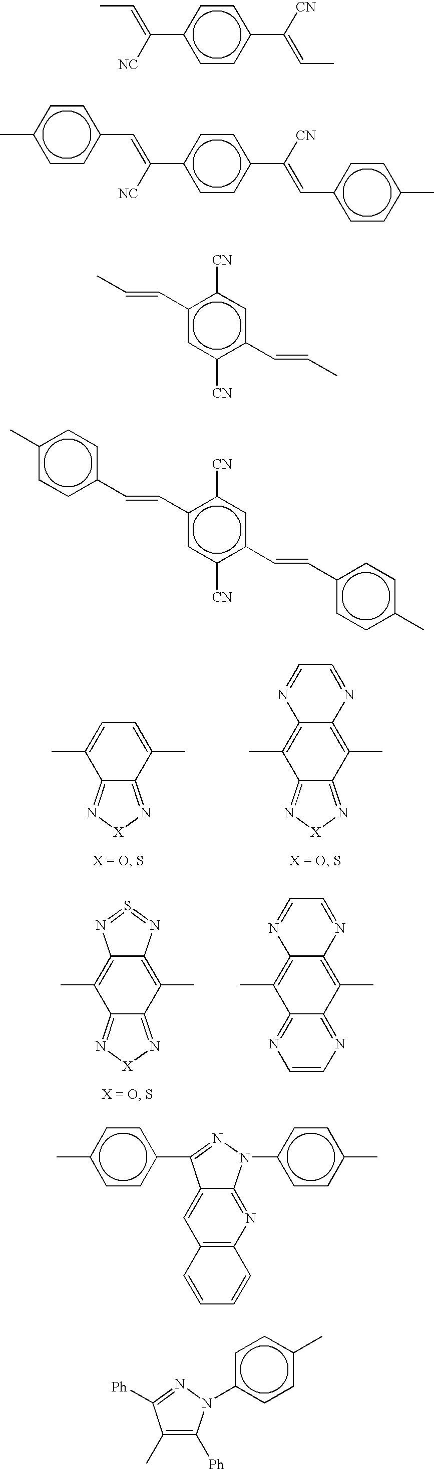

These electron transporting RMUs may bear a variety of substituents so long as their presence does not significantly affect electron transporting properties adversely. Preferred substituents are C₁₋₂₀ alkyls, C₆₋₂₀ aryls and alkylaryls optionally substituted with C₆₋₁₂ alkoxys and C₆₋₁₂ aryloxys. Particularly effective are RMUs derived from perfluorobiphenyl, quinoxalines, cyano-substituted olefins, oxadiazole, and benzothiadiazoles.

In another preferred embodiment, the co-polymers comprise at least 15 percent of RMUs of formula III, and at least 10 percent of two or more of the exemplified electron transporting RMUs. Most preferably the co-polymers comprise at least 20 percent of RMUs of formula III and at least 20 percent of two or more of the exemplified electron transporting RMUs. The ratio of the electron transporting RMUs may vary without limits so long as the combined percentage in the copolymer remains within the specified range. With respect to the electron transporting RMUs in the co-polymers of the invention, there is no restriction that they must all belong to the same chemical type. A co-polymer of the invention, may, for example, contain RMUs of the cyano-olefin type, RMUs of the oxadiazole type and RMUs of the condensed polynuclear aromatic type.

In a further preferred embodiment, copolymers of the invention preferably comprise at least 10 percent of RMUs of formula III and at least 1 percent of one or more hole transporting RMUs and at least 1 percent of one or more electron-transporting RMUs. Hole transporting RMUs and electron transporting RMUs are selected from among those already defined above. More preferably copolymers, of this embodiment comprise at least 15 percent of RMUs of formula III and at least 5 percent of one or more electron-transporting RMUs. Most preferably co-polymers of this embodiment comprise at least 20 percent of RMUS of formula III and at least 10 percent of one or more hole transporting RMUs and at least 10 percent of one or more electron-transporting RMUs. The ratio of the various hole transporting RMUs may vary without limits so long as the combined percentage in the co-polymer remains within the specified range. With respect to the hole transporting RMUs in the co-polymers of the invention, there is no restriction that they must all belong to the same chemical type. A co-polymer of the invention may, for example, contain RMUs of the silanyl type, RMUs of the thiophene, type and RMUs of the tertiary amine type. Similarly, with respect to the electron transporting RMUs in the co-polymers of the invention, there is no restriction that they must all belong to the same chemical type. A copolymer of the invention may, for example, contain RMUs of the cyano-olefin type, RMUs of the oxadiazole type and RMUs of the condensed polynuclear aromatic type.

In yet another preferred embodiment, co-polymers of the invention comprise at least 10 percent of RMUs of formula III, at least 1 percent of one or more RMUs derived independently in each occurrence from benzene, naphthalene, and biphenylene optionally substituted with C₁₋₁₂ alkyl/alkoxy and C₆₋₁₀ aryl/aryloxy (hereinafter referred to as arylene RMUs), and at least 1 percent of one or more RMUs selected from among the hole transporting and electron transporting RMUs defined above. Preferably co-polymers of this embodiment comprise at least 15 percent of RMUs of formula III, at least 5 percent of one or more arylene RMUs, and at least 1 percent of one or more RMUs selected from among the hole transporting and electron transporting RMUs defined above. Most preferably co-polymers of this embodiment comprise at least 20 percent of RMUs of formula III, at least 10 percent of one or more arylene RMUS, and at least 5 percent of one or more RMUs selected from among the hole transporting and electron transporting RMUs defined above. The ratio of the various arylene RMUs may vary without limits so long as the combined percentage in the copolymer remains within the specified range.

Incorporation of arylene RMUs can lead to modifications in the thermal, optical and electronic properties of the co-polymers.

Another aspect of this invention is related to polymer blends containing 1 to 99 percent of at least one carbazole-containing polymer of this invention. The remainder 1 percent to 99 percent of the blend is composed of one or more polymeric materials selected from among chain growth polymers such as polystyrene, polybutadiene, poly(methyl methacrylate), and poly(ethylene oxide); step-growth polymers such as phenoxy resins, polycarbonates, polyamides, polyesters, polyurethanes, and polyimides; and crosslinked polymers such as crosslinked epoxy resins, crosslinked phenolic resins, crosslinked acrylate resins, and crosslinked urethane resins. Examples of these polymers may be found in Preparative Methods of Polymer Chemistry, W. R. Sorenson and T W Campbell, Second Edition, Interscience Publishers (1968). Other polymers which may be used in the blends are conjugated polymers such as poly(phenylene vinylene), substituted poly(phenylene vinylene)s, substituted polyphenylenes and polythiophenes. Examples of these conjugated polymers are given by Greenham and Friend in Solid State Physics, Vol. 49, pp. 1-149 (1995).

The most preferred blend composition is composed of at least 51 percent of a conjugated polymer and at most 49 percent of a carbazole-containing polymer of this invention with the provision that the band-gap of the carbazole-containing polymer is narrower than the band-gap of the conjugated polymer. These most preferred compositions have high photoluminescent and electroluminescent efficiency. Such blends may be prepared by solution blending, or blending in the melt state.

The sixth aspect of the invention provides films formed from the oligomers, polymers and copolymers of the invention. Such films can be used in polymeric electroluminescent devices. Preferably, such films are used as light emitting layers or charge carrier transport layers. These oligomers, polymers and copolymers may also be used as protective coatings for electronic devices and as fluorescent coatings. The thickness of the coating or film is dependent upon the ultimate use. Generally, such thickness can be from 0.01 to 200 microns. In that embodiment wherein the coating is used as a fluorescent coating, the coating or film thickness is from 50 to 200 microns. In that embodiment where the coatings are used as electronic protective layers, the thickness of the coating can be from 5 to 20 microns. In that embodiment where the coatings are used in a polymeric light-emitting diode, the thickness of the layer formed is 0.05 to 2 microns. The oligomers of the invention form good pinhole- and defect-free films. Such films can be prepared by means well known in the art including spin-coating, spray-coating, dip-coating, roller-coating and doctor blade coating. Such coatings are prepared by a process comprising applying a composition to a substrate and exposing the applied composition to conditions such that a film is formed. The conditions which form a film depend upon the application technique and the reactive end groups of the aryl moiety. In a preferred embodiment, the composition applied to the substrate comprises the carbazole oligomer, polymer, or co-polymer dissolved in a common organic solvent. Preferably, the solution contains from 0.1 to 10 weight percent of the oligomer, polymer, or co-polymer. For thin coatings, it is preferred that the composition contains from 0.5 to 5.0 percent by weight of the oligomer, polymer, or co-polymer. This composition is then applied to the appropriate substrate by the desired method and the solvent is allowed to evaporate. Residual solvent may be removed by vacuum and/or by heat. If the solvent is low boiling, then low solution concentrations, for example, 0.1 to 2 percent, are desired. If the solvent is high boiling, then high concentrations, for example 3 to 10 percent, are desired. After removal of the solvent, the coating is then exposed to the necessary conditions to cure the film, if needed, to prepare a film having a high solvent and heat resistance. The films are preferable substantially uniform in thickness and substantially free of pinholes. Preferably, the films are cured when exposed to temperatures of 100 degree C. or greater, more preferably 150 degree C. and most preferably 200 degree C. or greater. Preferably, the films cure at a temperature of 300 degree C. or less.

In the preparation of the films, the composition may further comprise a catalyst suitable to facilitate or initiate the curing of the films. Such catalysts are well known in the art, for instance, for materials having ethylenic unsaturation, a free radical catalyst may be used. For carbazole moieties with glycidyl ethers as end-groups, ureas or imidazoles may be used. In the preparation of films from carbazoles with glycidyl ether aryl-terminal moieties, such material may be reacted with commonly known curing agents which facilitate crosslinking. Among preferred curing agents are tetrahydrophthalic anhydride, nadic anhydride and maleic anhydride.

In another embodiment, the carbazole oligomers, polymers, or co-polymers may be partially cured. This is known as B-staging. In such embodiment, the carbazoles and their oligomers or polymers thereof are exposed to conditions such that a portion of the reactive materials cure and a portion of the reactive materials do not cure. This is commonly used to improve the handleability of such a resin and can facilitate the preparation of the films. Such B-staged material can thereafter be used to prepare coatings by the means disclosed above. Preferably, 10 mole percent or greater of the reactive moieties are reacted. Preferably, 50 mole percent or less of the reactive moieties are reacted.

The seventh aspect of the invention relates to organic electroluminescent (EL) devices, and more particularly to light emitting diodes, comprising one or more of the polymers and/or co-polymers of the invention wherein the polymers and/or copolymers are present as single-layer films, or as multiple-layer films, whose combined thickness is in the range of 10 nm to 1000 nm, preferably in the range of 25 nm to 500 nm, most preferably in the range of 50 nm to 300 nm. When two or more polymers or co-polymers are used, they may be deposited separately as distinct layers or deposited as one layer from a solution containing a blend of the desired polymers or co-polymers each designed for a specific function.

An organic EI device typically consists of an organic film sandwiched between an anode and a cathode such that when a positive bias is applied to the device, holes are injected into the organic film from the anode, and electrons are injected into the organic film from the cathode. The combination of a hole and an electron may give rise to an exciton which may undergo radiative decay to the ground state by liberating a photon. The anode and the cathode may be made of any materials and in any structure known in the art. The anode is preferably transparent. In practice the anode is commonly a mixed oxide of tin and indium for its conductivity and transparency. The mixed oxide (ITO) is deposited on a transparent substrate such as glass or plastic so that the light emitted by the organic film may be observed.

Since holes are injected from the anode, the layer next to the anode heeds to have the functionality of transporting holes. Similarly, the layer next to the cathode needs to have the functionality of transporting electrons. In many instances, the hole-(electron) transporting layer also acts as the emitting layer. In some instances one layer can perform the combined functions of hole and electron transport and light emission. The individual layers of the organic film may be all polymeric in nature or combinations of films of polymers and films of small molecules deposited by thermal evaporation. It is preferred that the total thickness of the organic film be less than 1000 nanometers (nm). It is more preferred that the total thickness be less than. 500 nm, It is most preferred that the total thickness be less than 300 nm. One embodiment of the instant invention is EL devices whose organic film comprises at least one of the polymers or co-polymers of this invention.

The ITO-glass which serves as the substrate and the anode nay be used for coating after the usual cleaning with detergent, organic solvents and UV-ozone treatment. It may also be first coated with a thin layer of a conducting substance to facilitate hole injection. Such substances include copper phthalocyanine, polyaniline and poly (3,4-ethylenedioxy-thiophene) (PEDT); the last two in their conductive forms by doping with a strong organic acid, e.g., poly(styrenesulfonic acid). It is preferred that the thickness of this layer be 200 nm or less; it is more preferred that the thickness be 100 nm or less.

In the cases where a hole-transporting layer is used, the polymeric arylamines described in U.S. patent application Ser. No. 08/606,180 filed on Feb. 23, 1996; U.S. patent application Ser. No. 08/696,280 filed on Aug. 13, 1996; and U.S. patent application Ser. No. 08/696,281 filed on Aug. 13, 1996 may be used, all of which are hereby incorporated by reference. Other known hole-conducting polymers, such as polyvinylcarbazole, may also be used. The resistance of this layer to erosion by the solution of the polymer film which is to be applied next is obviously critical to the successful fabrication of multi-layer devices. For example, if the next polymer film is applied as a xylene or toluene solution, the hole-transporting layer needs to be insoluble in these solvents. The thickness of this layer may be 500 nm or less, preferably 300 nm or less, most preferably 150 nm or less.

In the case where an electron-transporting layer is used, it may be applied either by thermal evaporation of low molecular weight materials or by solution coating of a polymer with a solvent that would not cause significant damage to the underlying film.

Examples of low molecular weight materials include the metal complexes of 8-hydroxyquinoline (as described by Burrows et al. In Applied Physics Letters, Vol. 64, pp. 2718-2720 (1994), metallic complexes of 10-hydroxybenzo/(h)quinoline (as described by Hamada et al. in chemistry Letters, pp. 906-906 (1993)), 1,3,4-oxadiazoles (as described by Hamada et al. in Optoelectronics—Devices and Technologies, Vol. 7, pp. 83-93 (1992)), 1, 3, 4-triazoles (as described by Kido et al. in Chemistry Letters, pp. 47-48 (1996)), and dicarboximides of perylene (as described by Yoshida et al. in Applied Physics Letters, Vol 69, pp 734-736 (1996)).

Polymeric electron-transporting material are exemplified by 1,3,4-oxadiazole-containing polymers (as described by Li et al. in Journal of Chemical Society, pp. 2211-2212 (1995), by Yang and Pei in Journal of Applied Physics, Vol 77, pp. 4807-4809 (1995)), 1, 3, 4-triazole-containing, polymers (as described by Strukelj et al. in Science, Vol. 267 pp. 1969-1972 (1995)), quinoxaline-containing polymers (as described by Yamamoto et al. in Japan Journal of Applied Physics, Vol. 33, pp. L250-L253 (1994), O'Brien et al. in Synthetic Metals, Vol. 76, pp. 105-108(1996)) and cyano-PPV (as described by Weaver et al. in Thin Solid Films, Vol 273, pp. 39-47 (1996)). The thickness of this layer may be 500 nm or less, preferably 300 nm or less most preferably 150 nm or less.

The metallic cathode may be deposited either by thermal evaporation or by sputtering. The thickness of the cathode may be from 100 nm to 10,000 nm. The preferred metals are calcium, magnesium, indium and aluminium. Alloys of these metals may also be used. Alloys of aluminium containing 1 to 5 percent of lithium and alloys of magnesium containing at least 80 percent of magnesium are preferred.

The EL devices of this invention emit light when subjected to an applied voltage of 50 volt or less with luminance efficiency as high as 3.5 Cd/A.

In a preferred embodiment, the electroluminescent device comprises at least one hole-transporting polymer film and a light-emitting polymer film comprised of a polymer or co-polymer of the invention, arranged between an anode material and a cathode material such that under an applied voltage, holes are injected from the anode material into the hole-transporting polymer film and electrons are injected from the cathode material into the light-emitting polymer films when the device is forward biased, resulting in light emission from the light-emitting layer. In another preferred embodiment, layers of hole-transporting polymers are arranged so that the layer closest to the anode has the lower oxidation potential, with the adjacent layers having progressively higher oxidation potentials. By these methods, electroluminescent devices having relatively high light output per unit voltage may be prepared.

The term “hole-transporting polymer film” as used herein refers to a layer of a film of a polymer which when disposed between two electrodes to which a field is applied and holes are injected from the anode, permits adequate transport of holes into the emitting polymer. Hole-transporting polymers typically are comprised of triarylamine moieties. The term “light-emitting polymer film” as used herein refers to a layer of a film of a polymer whose excited states can relax to the ground state by emitting photons, preferably corresponding to wavelengths in the visible range, The term “anode material” as used herein refers to a semi-transparent, or transparent, conducting film with a work function between 4.5 electron volts (eV) and 5.5 eV. Examples are oxides and mixed oxides of indium and tin, and gold. The term “cathode material” as used herein refers to a conducting film with a work function between 2.2 eV and 4.5 eV. Examples are lithium, calcium, magnesium, indium, silver, aluminium, or blends and alloys of the above.

In another embodiment, the invention provides a photocell comprising one or more of the polymers and/or co-polymers of the invention wherein the polymers and/or co-polymers are present as single-layer films or as multiple-layer films, whose combined thickness is in the range of 10 mm to 1000 nm, preferably in the range of 25 nm to 500 nm, most preferably in the range of 50 nm to 300 nm. The polymer or co-polymer films may be formed as described above. By photocells is meant a class of optoelectronic devices which can convert incident light energy into electrical energy. Examples of photocells are photovoltaic devices, solar cells, photodiodes, and photodetectors. A photocell generally comprises a transparent or semi-transparent first electrode deposited on a transparent substrate. A polymer film is then formed onto the first electrode which is, in turn, coated by a second electrode. Incident light transmitted through the substrate and the first electrode is converted by the polymer film into excitons which can dissociate into electrons and holes under the appropriate circumstances, thus generating an electric current.

In a still further embodiment, the invention provides a metal-insulator-semiconductor field effect transistor comprising one or more of the polymers and/or co-polymers of the invention (serving as the semi-conducting polymer) deposited onto an insulator wherein the polymers or co-polymers are present as single-layer films or as multiple-layer films whose combined thickness is in the range of 10 nm to 1000 nm, preferably in the range of 25 nm to 500 nm, most preferably in the range of 50 nm to 300 nm. The co-polymer films may be formed as previously described. Two electrodes (source and drain) are attached to the semi-conducting polymer and a third electrode (gate) onto the opposite surface of the insulator. If the semi-conducting polymer is hole tranporting (that is, the majority carriers are positive holes), then applying a negative DC voltage to the gate electrode induces an accumulation of holes near the polymer-insulator interface, creating a conduction channel through which electric current can flow between the source and the drain. The transistor is in the “on” state. Reversing the gate voltage causes a depletion of holes in the accumulation zone and cessation of current. The transistor is in the “off” state. If the semi-conducting polymer is electron transporting (that is, the majority carriers are electrons), then applying a positive DC voltage to the gate electrode induces a deficiency of holes (accumulation of electrons) near the polymer-insulator interface, creating a conduction channel through which electric current can flow between the source and the drain.

The invention is illustrated by the following examples. Unless otherwise stated, all parts and percentages are by weight.

### EXAMPLE 1

### EXAMPLE 1

### 2, 7-dimethyl-carbazole,

A solution of 2,2′-diamino-4,4′-dimethyl-biphenyl (5.55 g, 26.14 mmol) and dodecyl benzenesulfonic acid (17.19 g, 52.70 mmol) in 5-t-butyl-m-xylene (170 cm³) was refluxed for 70 h. The solution was evaporated to dryness on heating in vacuo. The residue was purified by column chromatography on silica gel—(toluene/hexane 1/4) to give 2,7-dimethyl-carbazole as a colourless powder (4.85 g, 95% yield). The purity of the product was confirmed by HPLC >99% purity. Thin layer chromatography (a single spot on silica-gel plate (Rf=0.63)-toluene), m. p.: 280-281° C. (lit. 280-281° C.).

### EXAMPLE 2

### 2,7-dichloro-carbazole,

A solution of 2,2′-diamino-4,41-dichlorobiphenyl (0.76 g, 3.00 mmol) and dodecylbenzenesulfonic acid (1.94 g, 3.00 mmol) in 5-t-butyl-rr-xylene (20 cm³) was refluxed for 20 h. The solution was evaporated to dryness on heating in vacuo. The residue was purified by column chromatography on silica gel—(toluene/hexane 3/7) to give 2,7-dichlorocarbazole as a colourless powder (0.67 g, 95% yield). The purity of the product was confirmed by HPLC >99% purity. Thin layer chromatography (a single spot on silica-gel plate (Rf=0.6)—ethyl acetate/hexane 1/4), m, p. 210-211° C. (lit. 204° C.).

### EXAMPLE 3

### 2,7-Dibromo-carbazole

A solution of 2,2′-diamino-4,4′-dibromo-biphenyl (0.51 g, 1.30 mmol) and dodecylbenzenesulfonic acid (0.97 g, 3.00 mmol) in 5-t-butyl-m-xylene (10 cm³) was refluxed for 24 h. The solution was evaporated to dryness on heating in vacuo. The residue was purified by column chromatography on silica gel—toluene/hexane (3/7) to give 0,2,7-dibromocarbazole as a colourless powder (0.47 g, 1.45 mmol, 96 t yield). The purity of the product was confirmed by HPLC (>99% purity). Thin layer chromatography (a single spot on silica-gel plate (R 0.50)—ethyl acetate/hexane 1/4). GC-MS (m/z) 323, 325, 327 (M⁺). M. p.: 224-225° C. (lit. 198-203° C.).

### EXAMPLE 4

### 2,7-Dibromo-3,6-dimethyl-carbazole

A solution of 2,2′-diamino-4,4′-dibromo-5,5′-dimethyl-biphenyl (5.00 g, 13.51 mmol) and dodecylbenzenesulfonic acid (8.73 g, 27.02 mmol) in 5-t-butyl-m-xylene (60 cm³) was refluxed for 48 h. The solution was evaporated to dryness on heating in vacuo. The residue was purified by column chromatography on silica gel—toluene/hexane (1/8) to give 2,7-dibromo-3,6-dimethyl-carbazole as a colourless powder (4.06 g, 85% yield). The purity of the product was confirmed by HPLC (>999 purity). Thin layer chromatography (a single spot on silica-gel plate (Rf=0.60)—ethyl acetate/hexane 1/4). GC-MS (m/z): 351, 353, 355 (M⁺).

### EXAMPLE 5

### 2,7-dibromo-9-(2-ethylhexyl)-carbazole,

A mixture of 2,7-dibromocarbazole (4.56 g, 14.0 mmol)), 1-bromo-2-ethylhexane (2.97 g, 15.4 mmol), tetra-n-butylammonium hydrogen sulfide (0.14 g, 0.42 mmol), and NaOH (0.84 g, 21.0 mmol) (ground before use) in acetone (HPLC grade) (30 cm³) was refluxed for 9 h. After the reaction, the acetone was removed in vacuo and the residue was extracted with toluene (300 cm³). The toluene solution was washed with a saturated NaCl aqueous solution (3×200 cm³), dried over MgSO₄, and evaporated to dryness in vacuo. The residue was purified by column chromatography on silica gel 60—hexane to give 2,7-dibromo-9-(2-ethylhexyl)carbazole as a colourless powder (5.49 g, 90 t yield). The purity of the product was confirmed by HPLC (>99% purity). The product gave a single spot on TLC (Rf=0.85, hexane), m. p. 95-97° C. GC-MS (m/z) 435, 437, 439 (M⁺).

### EXAMPLE 6

### 2,7-dibromocarbazole-9-dodecyl-carbazole

A mixture of 2,7-dibromocarbazole (7.80 g 24.0 mmol), 1-bromododecane, (8.97 g, 36.0 mmol), tetra-n-butylammonium hydrogen sulfide (0.49 g, 1.44 mmol), and NaOH (1.92 g, 48.0 mmol) (ground before use) in acetone (HPLC grade) (30 cm³) was refluxed for 5 h. After the reaction, the acetone was removed in vacuo and the residue was extracted with toluene (400 cm³). The toluene solution was washed with a saturated NaCl aqueous solution (3×300 cm³), dried over MgSO₄, and evaporated to dryness in vacuo. The residue was purified by column chromatography on silica gel 60—hexane to give 2,7-dibromo-9-dodecyl carbazole as a colourless powder (10.28 g, 87% yield). The purity of the product was confirmed by HPLC (>99% purity). The product gave a single spot on TLC (Rf=0.82, hexane), m. p. 78-79° C. GC-MS (m/z); 491, 493, 495 (M⁺).

### EXAMPLE 7

### 2,7-dibromo-9-hexadecyl-carbazole

A mixture of 2,7-dibromocarbazole (7.80 g 24.0 mmol), 1-bromohexadecane, (10.99 g, 36.0 mmol), tetra-n-butylammonium hydrogen sulfide (0.49 g, 1.44 mmol), and NaOH (1.92 g, 48.0 mmol) (ground before use) in acetone (HPLC grade) (50 cm³) was refluxed for 5 h. After the reaction, the acetone was removed in vacuo and the residue was extracted with toluene (400 cm³). The toluene solution was washed with a saturated NaCl aqueous solution (3×300 cm³), dried over MgSO₄, and evaporated to dryness in vacuo. The residue was purified by column chromatography on silica gel 60—hexane to give 2,7-dibromo-9-hexadecylcarbazole as a colourless powder (11.32 g, 86% yield). The purity of the product was confirmed by HPLC (>99% purity). The product gave a single spot on TLC (Rf=0.83, hexane), m. p.: 85-86° C. GC-MS (m/z): 547, 549, 551 (M⁺).

### EXAMPLE 8

### 2,7-dibromo-9-(2-hexyldecyl)-carbazole

A mixture of 2,7-dibromocarbazole (7.80 g 24.0 mmol), 1-bromo-2-hexyldecane, (10.99 g, 36.0 mmol), tetra-n-butylammonium hydrogen sulfide (0.49 g, 1.44 mmol), and NaOH (1.92 g, 48.0 mmol) (ground before use) in acetone (HPLC grade) (50 cm³) was refluxed for 5 h. After the reaction, the acetone was removed in vacuo and the residue was extracted with toluene (400 Cm³) The toluene solution was washed with a saturated NaCl aqueous solution (3×300 cm³), dried over MgSO₄, and evaporated to dryness in vacuo. The residue was purified by column chromatography on silica gel 60—hexane to give 2,7-dibromo-9-(2-hexyldecyl)carbazole as a pale yellow oil (12.42 g, 94% yield). The purity of the product was confirmed by HPLC (>99% purity). The product gave a single spot on TLC (Rf=0.83, hexane). GC-MS (m/z): 547, 549, 551 (M⁺).

### EXAMPLE 9

### 2,7-Dibromo-3,6-dimethyl-9-(2-hexyldecyl)-carbazole

A mixture of 2,7-dibromo-3,6-dimethyl-carbazole (3.00 g 8.50 mmol), 1-bromo-2-hexyldecane, (3.89 g, 12.75 mmol), tetra-n-butylammonium hydrogen sulfide (0.17 g, 0.51 mmol), and NaOH (0.68 g, 17.0 mmol) (ground before use) in acetone (HPLC grade) (30 cm³) was refluxed for 0.8 h. After the reaction, the acetone was removed in vacuo and the residue was extracted with toluene (300 cm³). The toluene solution was washed with a saturated NaCl aqueous solution (3×200 cm³), dried over MgSO₄, and evaporated to dryness in vacuo. The residue was purified by column chromatography on silica gel 60—hexane to give 2,7-dibromo-3,6-dimethyl-9-(2-hexyldecyl)-carbazole as a clear oil (4.61 g, 94% yield). The purity of the product was confirmed by HPLC (>99% purity). The product gave a single spot on TLC (Rf=0.83, hexane). GC-MS (m/z): 575, 577, 579 (M⁺).

### EXAMPLE 10

### 2,7-dichlorocarbazole-9-dodecyl-carbazole

A mixture of 2,7-dichlorocarbazole (5.90 g, 25.0 mmol), 1-bromododecane, (9.35 g, 37.5 mmol), tetra-n-butylammonium hydrogen sulfide (0.51 g, 1.50 mmol), and NaOH (1.98 g, 50.00 mmol) (ground before use) in acetone (HPLC grade) (35 cm³) was refluxed for 9 h. After the reaction, the acetone was removed in vacuo and the residue was extracted with toluene (400 cm³). The toluene solution was washed with a saturated NaCl aqueous solution (3×300 cm³), dried over MgSO₄/and evaporated to dryness in vacuo. The residue was purified by column chromatography on silica gel 60—hexane to give 2,7-dichloro-9-dodecyl carbazole as a colourless powder (8.69 g, 86% yield). The purity of the product was confirmed by HPLC (>99% purity). The product gave a single spot on TLC (Rf=0.84, hexane), m. p. 70-71° C.

### EXAMPLE 11

### 2,7-dichloro-9-hexadecyl-carbazole

A mixture of 2,7-dichlorocarbazole (6.00 g, 25.41 mmol), 1-bromohexadecane, (11.64 g, 38.12 mmol), tetra-n-butylammonium hydrogen sulfide (0.50 g, 1.46 mmol), and NaOH (1.95 g, 48.79 mmol) (ground before use) in acetone (HPLC grade) (50 cm³) was refluxed for 9 h. After the reaction, the acetone was removed in vacuo and the residue was extracted with toluene (400 cm³). The toluene solution was washed with a saturated NaCl aqueous solution (3×300 cm³), dried over MgSO₄, and evaporated to dryness in vacuo. The residue was purified by column chromatography on silica gel 60—hexane to give 2,7-dichloro-9-hexadecylcarbazole as a colourless powder (10.18 g, 87% yield). The purity of the product was confirmed by HPLC (>99% purity). The product gave a single spot on TLC (Rf=0.83, hexane), m. p.: 82-83° C.

### EXAMPLE 12

### 2,7-dichloro-9-(2-hexyldecyl)-carbazole

A mixture of 2,7-dichlorocarbazole (6.00 g, 25.41 mmol), 1-bromo-2-hexyldecane, (11.64 g, 38.12 mmol), tetra-n-butylammonium hydrogen sulfide (0.50 g, 1.46 mmol), and NaOH (1.95 g, 48.79 mmol) (ground before use) in acetone (HPLC grade) (50 cm³) was refluxed for 9 h. After the reaction, the acetone was removed in vacuo and the residue was extracted with toluene (400 cm³). The toluene solution was washed with a saturated NaCl aqueous solution (3×300 c dried over MgSO₄, and evaporated to dryness in vacuo. The residue was purified by column chromatography on silica gel 60—hexane to give 2,7-dichloro-9-(2-hexyldecyl)carbazole as a clear oil (10.53 g, 90% yield). The purity of the product was confirmed by HPLC (>99% purity). The product gave a single spot on TLC (Rf=0.83, hexane).

### EXAMPLE 13

### 2,7-Bis (4,4,5,5-tetramethyl-1, 3, 2-dioxaborolan-2-yl)-9-hexadecyl-carbazole

To a solution of 2,7-dibromo-9-hexadecyl-carbazole (4 g, 7.28 mmol) in THF (60 cm³) at −78° C. was added 9.55 cm³ of 1.6 M solution of n-butyllithium in hexanes (15.3 mmol). The mixture was stirred at −78° C. for 30 min, then allowed to warm gradually to 0° C. and kept at 0° C. for 15 min, and cooled again to −78° C. 2-Isopropoxy-4,4,5,5-tetramethyl-1,3,2-dioxaborolane (3.20 g, 17.18 mmol) was then added to the solution, and the resulting mixture was allowed to warm gradually to room temperature and stirred for 24 h. The mixture was poured into water (300 cm³) and extracted with diethyl ether (3×300 cm³). The organic extracts were washed with a saturated NaCl aqueous solution (3×300 cm³) and dried over MgSO₄, and evaporated to dryness in vacuo. The residue was purified by column chromatography on silica gel—(ethyl acetate/hexane 1/10) to give 2,7-bis(4,4,5,5-tetramethyl-1/3,2-dioxaborolan-2-yl)-9-hexadecyl-carbazole as a pale yellow solid (3.14 g, 67% yield). The purity of the product was confirmed by HPLC >99% purity. Thin layer chromatography (a single spot on silica-gel plate (Rf=0.6) ethyl acetate/hexane 1/10).

### EXAMPLE 14

### 2,7-Bis (boronic acid)-9-hexadecyl-carbazole

To a solution of 2,7-dibromo-9-hexadecyl-carbazole (4 g, 7.28 mmol) in THF (60 cm³) at −78° C. was added 9.55 cm³ of 1.6 M solution of n-butyllithium in hexanes (15.3 mmol). The mixture was stirred at −78° C. for 30 min, then allowed to warm gradually to 0° C. and kept at 0° C. for 15 min, and cooled again to −78° C. Triisopropoxyborane (6.46 g, 34.36 mmol) was then added to the solution, and the resulting mixture was allowed to warm gradually to room temperature and stirred for 24 h. The mixture was poured into an aqueous 1 M HCl solution (300 cm³) and extracted with diethyl ether (3×300 cm³). The organic extracts were washed with water (200 cm³) a saturated NaCl aqueous solution (3×300 cm³) and dried over MgSO₄, and evaporated to dryness in vacuo. The residue was purified by column chromatography on silica gel—(ethyl acetate/hexane 1/10) to give 2,7-bis(4,4,5,5-tetramethyl-1,3,2-dioxaborolan-2-yl)-9-hexadecyl-carbazole as a pale yellow solid (2.44 g, 70% yield). The purity of the product was confirmed by HPLC >99% purity.

### EXAMPLE 15

### 2,7-Bis(tri-(n-butyl)stannyl)-9-(2-hexyldecyl)-carbazole

To a solution of 2,7-dibromo-9-(2-hexyldecyl)-carbazole (3 g, 5.46 mmol) in THF (40 cm³) at −78 IC was added 7.20 cm³ of 1.6 M solution of n-butyllithium in hexanes (11.48 mmol). The mixture was stirred at −78 IC for 30 min, then allowed to warm gradually to 0 IC and kept at 0° C. for 15 min, and cooled again to −78° C. Tri(n-butyl) tin chloride (4.20 g, 12.90 mmol) was then added to the solution, and the resulting mixture was allowed to warm gradually to room temperature and stirred for 24 h. The mixture was then poured onto ice, and the products extracted into diethyl ether (3×200 cm³) and washed with water (2×200 cm³), saturated aqueous copper sulphate (300 cm³), saturated sodium hydrogen carbonate (200 cm³) and water again (300 cm³). The organic extracts were then dried over MgSO₄, and evaporated to dryness in vacuo to leave an orange red oil. The oil was separated by flash chromatography using hexane on pre-treated silica (washed with triethylamine then hexane) to afford 2,7-bis(tri-(n-butyl)stannyl)-9-(2-hexyldecyl)-carbazole as a pale yellow oil. Anal. calcd. for C₅₂H₉₃NSn_{z}: C, 64.41; H, 9.67; N, 1.44. Found: C, 64.38; H, 9.65; N, 1.41.

### EXAMPLE 16

### Poly(9-dodecylcarbazole)-2,7-diyl

2,7-dibromo-9-dodecylcarbazole (1.48 g, 3.00 mmol) magnesium (turnings) (80.2 mg, 3.30 mmol), (2,2′-bipyridine) dichloropalladium(II) (20.0 mg, 0.060 mmol), and THF (15 cm³) was placed in a sealed glass tube and heated at 120° C. with stirring for 72 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly(9-dodecylcarbazole)-2,7-7-diyl as a green powder (0.81 g, 81% yield). GPC: Mw=10,200; Mn=3,800; Mw Mn=2.7. Soxhlet extraction with hexane over 24 h affords 0.47 g of the polymer as a gray olive powder. GPC: Mw=8600; Mn=6300; Mw/Mn=1.4.

### EXAMPLE 17

### 1.1 Poly(9-hexadecylcarbazole)-2,7-diyl

2,7-dibromo-9-hexadecylcarbazole (1.43 g, 2.60 mmol) magnesium (turnings) (69.5 mg, 2.86 mmol), (2,2′-bipyridine) dichloropalladium(II) (17.3 mg, 0.052 mmol), and THF (15 cm³) was placed in a sealed glass tube and heated at 120° C. with stirring for 504 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly(9-hexadecylcarbazole)-2,7-diyl as a green powder (0.84 g, 83% yield). GPC: Mw=14,000; Mn 4,800; Mw/Mn=3.0. Soxhlet extraction with hexane over 24 h affords 0.62 g of the polymer as an olive powder. GPC: Mw=10700; Mn=6800; Mw/Mn=1.6.

### EXAMPLE 18

### Poly(9-(2-hexyldecyl)carbazole)-2,7-diyl

2,7-dibromo-9-(2-hexyldecyl)carbazole (4.29 g, 7.80 mmol) magnesium (turnings) (208.5 mg, 8.56 mmol), (2,2′-bipyridine) dichloropalladium(II) (51.9 mg, 0.156 mmol), and THF (30 cm³) were placed in a sealed glass tube and heated at 120° C. with stirring for 72 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly (9-(2-hexyldecyl) carbazole)-2,7-diyl as a yellow powder (2.82 g, 93% yield). GPC: Mw=19,500; Mn=5, 100; Mw/Mn=3.8. Soxhlet extraction with hexane over 24 h affords 1.68 g of the polymer as a deep yellow powder. GPC: Mw=21500; Mn=8200; Mw/Mn=2.6.

### EXAMPLE 19

### Poly(9-(2-hexyldecyl)-carbazole)-2,7-diyl using Suzuki-type coupling

2,7-Dibromo-0.9-(2-hexyldecyl)-carbazole (1.8 g, 3.28 mmol), 2,7-bis (4,4, 5,5-tetramethyl-1,3,2-dioxaborolan-2-yl)-9-(2-hexyldecyl)-carbazole (2.11 g, 3.28 mmol) and tetrakis(triphenylphosphine) palladium(0) (80 mg, 0.07 mmol) were dissolved in THF (16 cm³) under an inert atmosphere. A de-oxygenated 2M aqueous. K₂CO₃ solution 10.5 cm³) was then added and the resulting mixture was vigorously stirred and heated at 80° C. for 72 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly(9-(2-hexyldecyl)carbazole)-2,7-diyl as a yellow-brown powder. Soxhlet extraction with acetone over 24 h affords 1.92 g of the polymer as a deep yellow powder. (75% yield). GPC: Mw=29,000; Mn=12,600; Mw/Mn=2.3.

### EXAMPLE 20

### Poly(9-(2-hexyldecyl)-carbazole)-2,7-diyl using Stille-type coupling

2,7-Dibromo-9-(2-hexyldecyl)-carbazole (1.8 g, 3.28 mmol), 2,7-bis (tri-(n-butyl)stannyl)-9-(2-hexyldecyl)-carbazole (3.18 g, 3.28 mmol) and tetrakis(triphenylphosphine) palladium(0) (80 mg, 0.07 mmol) were dissolved in toluene (20 cm³) under an inert atmosphere. The resulting mixture was heated to reflux and stirred for 48 h. Upon cooling the reaction mixture was poured into methanol under an inert nitrogen atmosphere. The precipitate was filtered off, then dissolved in chloroform. The insoluble materials in the chloroform solution were filtered off, and then the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly(9-(2-hexyldecyl)carbazole)-2,7-diyl as a yellow powder. Soxhlet extraction with acetone over 24 h affords 2.1 g of the polymer as a deep yellow powder. (82% yield). GPC: Mw=21,100; Mn=8,600, Mw/Mn=2.5.

### EXAMPLE 21

### Poly(9-(2-hexyldecyl)-carbazole)-2,7-diyl using Yamamoto-type Coupling

A mixture of bis (1,5-cyclooctadienyl)nickel (0) (0.97 g, 3.53 mmol), 2,2′-bipyridyl (0.55 g, 3.53 mmol) and 1,5-cyclooctadiene (0.38 g, 3.53 mmol), toluene (8 cm³) and DMF (8 cm³) was heated under an inert atmosphere to 80° C. for 30 min. A solution of 2,7-dibromo-9-(2-hexyldecyl)-carbazole (1.00 g, 1.82 mmol) and 4-bromotoluene (34 mg, 0.2 mmol) in degassed toluene (8 cm³) was then added to the mixture and the reaction was then maintained at 80° C. for 24 h. The polymer was then precipitated into a solvent mixture of methanol (150 cm³), acetone (150 cm³) and concentrated hydrochloric acid (150 cm³). The polymer was then subjected to soxhlet extraction with methanol over 24 h. It was then dissolved in chloroform and the insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly(9-(2-hexyldecyl)carbazole)-2,7-diyl as a yellow powder. Soxhlet extraction with acetone over 24 h affords 0.56 g of the polymer as a deep yellow powder. (79: yield). GPC: Mw=33,000; Mn=17,400; Mw/Mn=1.9.

### EXAMPLE 22

### Poly(3,6-dibromo-9-(2-hexyldecyl)-carbazole)-2,7-diyl

To a solution of poly(9-(2-hexyldecyl)carbazole)-2,7-diyl (Mw=21500; Mn=8200) (0.5 g, 1.28 mmol) in chloroform (50 cm³) was added a solution of N-bromosuccinimide (0.55 g, 3.10 mmol) in chloroform (50 cm³) in the dark. The solution was stirred at room temperature for 18 h and then heated to 50° C. for 2 h and the reaction mixture poured into a saturated NaHCO₃ solution (50 cm³). The organic layer was washed with water (5×50 cm³) and dried over MgSO₄. The solution was concentrated in vacuo and poured into methanol (400 cm³) The precipitate was re-precipitated from chloroform into methanol two more times. The precipitate was dried under vacuum to give poly(3,6-dibromo-9-(2-hexyldecyl)-carbazole)-2,7-diyl as a green powder (0.69 g, 98% yield based on 100% 3,6-substitution). GPC: Mw=26300; Mn=10200; Mw/Mn=2.6.

### EXAMPLE 23

### 1.2 Poly(3,6-dimethyl-9-(2-hexyldecyl)-carbazole)-2,7-diyl by direct polymerisation

2,7-Dibromo-3,6-dimethyl-9-(2-hexyldecyl)-carbazole (1.5 g, 2.60 mmol) magnesium (turnings) (69.5 mg, 2.86 mmol), (2,21-bipyridine) dichloropalladium(II) (17.3 mg, 0.052 mmol), and THF (15 cm³) was placed in a sealed glass tube and heated at 120° C. with stirring for 120 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly(3,6-dimethyl-9-(2-hexyldecyl)-carbazole)-2,7-diyl as a green powder (1.00 g, 92% yield). GPC: Mw=22,500; Mn=6,200; Mw/Mn 3.6. Soxhlet extraction with hexane over 24 h affords 0.62 g of the polymer as a deep green powder. GPC: Mw=24,000; Mn=9,300; Mw/Mn=2.6.

### EXAMPLE 24

### Poly(3,6-dimethyl-9-(2-hexyldecyl)-carbazole)-2,7-diyl by Polymer Homnologous Reaction

Methyl iodide (0.5 g, 3.52 mmol) was added to magnesium (turnings) (85.6 mg, 3.52 mmol) in THF (10 cm³). The resulting methyl magnesium iodide solution was added dropwise at 0° C. to a mixture of poly(3,6-dibromo-9-(2-hexyldecyl)-carbazole)-2,7-diyl (Mw=26300; Mn=10200) (0.3 g, 0.55 mmol) and 1,3-bis-(diphenylphosphino)-propane-dichloronickel(II) (15 mg, 0.027 mmol) in THF (10 cm³). The mixture was left to warm to room temperature and was then refluxed for 48 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly(3,6-dimethyl-9-(2-hexyldecyl)-carbazole)-2,7-diyl as a green powder (0.22 g, 96% yield). (The polymer shows the same NMR spectra as those from the polymer made by direct polymerisation of 2,7-dibromo-3,6-dimethyl-9-(2-hexyldecyl)-carbazole). GPC: Mw=27,500; Mn=10,600; Mw/Mn=2.6.

### EXAMPLE 25

### Poly{(2,2′-bithiophene)-5,5′-diyl-alt-co-(9-(2-hexyldecyl)-carbazole)-2,7-diyl)}

5,5′-Dibromo-2,2′-bithiophene (1.00 g, 3.09 mmol), 2,7-bis(4,4,5,5-tetramethyl-1,3,2-dioxaborolan-2-yl)-9-(2-hexyldecyl)-carbazole (1.98 g, 3.09 mmol) and tetrakis(triphenylphosphine) palladium(0) (92 mg, 0.08 mmol) were dissolved in THF (16 cm³) under an inert atmosphere. A de-oxygenated 2M aqueous K₂CO₃ solution (10.5 cm³) was then added and the resulting mixture was vigorously stirred and heated at 80° C. for 48 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly{(2,2′-bithiophene)-5,5′-diyl-alt-co-(9-(2-hexyldecyl)-carbazole)-2,7-diyl)} as a green powder. Soxhlet extraction with acetone over 24 h affords 1.36 g of the polymer as a dark green powder. (80% yield). GPC: Mw=28,800; Mn=13,100; Mw/Mn=2.2.

### EXAMPLE 26

### Poly{(2,5-bis (decyloxy)-benzene-1,4-diyl)-alt-co-(9-(2-hexyldecyl)-carbazole)-2,7-diyl)}

1,4-Dibromo-2,5-bis(decyloxy)-benzene (1.09 g, 2.00 mmol), 2,7-bis(4,4,5,5-tetramethyl-1,3,2-dioxaborolan-2-yl)-9-(2-hexyldecyl)-carbazole (1.28 g, 2.00 mmol) and tetrakis(triphenylphosphine) palladium(0) (60 mg, 0.052 mmol) were dissolved in THF (10 cm³) under an inert atmosphere. A de-oxygenated 2M aqueous K₂CO₃ solution 6.5 cm³) was then added and the resulting mixture was vigorously stirred and heated at 80° C. for 48 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give poly{(2,5-bis(decyloxy)-benzene-1,4-diyl)-alt-co-(9-(2-hexyldecyl)-carbazole)-2,7-diyl)} as a grayish-green powder. Soxhlet extraction with acetone over 24 h affords 1.27 g of the polymer as a green powder. (82% yield). GPC: Mw=23,300; Mn=11,100; Mw/Mn=2.1.

### EXAMPLE 27

### Poly{(9-(2-hexyldecyl)-carbazole)-2,7-diyl)-alt-co-(naphthalene-1,4-diyl)}

1,4-Dibromo-naphthalene (0.57 g, 2.00 mmol), 2,7-bis(4,4,5,5-tetramethyl-1,3,2-dioxaborolan-2-yl)-9-(2-hexyldecyl)-carbazole (1.28 g, 2.00 mmol) and tetrakis(triphenylphosphine) palladium(0) (60 mg, 0.052 mmol) were dissolved in THF (10 cm³ under an inert atmosphere. A de-oxygenated 2M aqueous K₂CO₃ solution 6.5 cm³) was then added and the resulting mixture was vigorously stirred and heated at 80° C. for 48 h. Upon cooling the reaction mixture was poured into methanol under an inert nitrogen atmosphere. The precipitate was filtered off, then dissolved in chloroform. The insoluble materials in the chloroform solution were filtered off, after which the filtrate was concentrated in vacuo. The concentrated chloroform solution was poured into methanol under inert nitrogen atmosphere, and the precipitate war filtered off. The precipitate was re-precipitated from chloroform into methanol under inert nitrogen atmosphere two more times. The precipitate was dried under vacuum to give poly{(9-(2-hexyldecyl)-carbazole)-2,7-diyl)-alt-co-(naphthalene-1,4-diyl)} as a green powder. Soxhlet extraction with acetone over 24 h affords 0.87 g of the polymer as a olive green powder. (84% yield). GPC: Mw=18,900; Mn=8,200; Mw/Mn=2.3.

### EXAMPLE 0.28

### Statistical copolymer comprising 85% of 2,7-linked-(9-(2-hexyldecyl)-carbazole) and 15% of 1,4-linked-naphthalene

A mixture of bis(1,5-cyclooctadienyl)nickel(0) (0.97 g, 3.53 mmol), 2,2′-bipyridyl (0.55 g, 3.53 mmol) and 1,5-cyclooctadiene (0.38 g, 3.53 mmol), toluene (8 cm³) and DMF (8 cm³) was heated under an inert atmosphere to 80° C. for 30 min. A solution of 2,7-dibromo-9-(2-hexyldecyl)-carbazole (0.85 g, 1.547 mmol), 1,4-dibromo-naphthalene (78 mg, 0.273 mmol) and 4-bromotoluene (34 mg, 0.2 mmol) in degassed toluene (8 cm³) was then added to the mixture and the reaction was then maintained at 80° C. for 24 h. The polymer was then precipitated into a solvent mixture of methanol (150 cm³), acetone (150 cm³) and concentrated hydrochloric acid (150 cm³). The polymer was then subjected to soxhlet extraction with methanol over 24 h. It was then dissolved in chloroform and the insoluble materials in the chloroform solution were filtered off, after which the filtrate was concentrated in vacuo The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give the statistical polymer as a yellow powder. Soxhlet extraction with acetone over 24 h affords 0.52 g of the polymer as a deep yellow powder. (82% yield). GPC; Mw=26,800; Mn=14,100; Mw/Mn=1.9.

### EXAMPLE 29

### Statistical copolymer comprising 85% of 2,7-linked-(9-(2-hexyldecyl)-3,6-dimethyl-carbazole) and 15% of 1,4-linked-na phthalene

A mixture of bis(1,5-cyclooctadienyl)nickel (0) (1.07 g, 3.88 mmol), 2,2′-bipyridyl (0.61 g, 3.88 mmol) and 1,5-cyclooctadiene (0.42 g, 3.88 mmol), toluene (8 cm³) and DMF (8 cm³) was heated under an inert atmosphere to 80° C. for 30 min. A solution of 2,7-dibromo-9-(2-hexyldecyl)-3,6-dimethyl-carbazole (0.98 g, 1.70 mmol), 1,4-dibromo-naphthalene (86 mg, 0.30 mmol) and 4-bromotoluene (37 mg, 0.22 mmol) in degassed toluene (8 cm³), was then added to the mixture and the reaction was then maintained at 80° C. for 24 h. The polymer was then precipitated into a solvent mixture of methanol (150 cm³), acetone (150 cm³) and concentrated hydrochloric acid (150 cm³. The polymer was then subjected to soxhlet extraction with methanol over 24 h. It was then dissolved in chloroform and the insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give the statistical polymer as a green powder. Soxhlet extraction with acetone over 24 h affords 0.63 g of the polymer as a deep green powder. (84% yield). GPC: Mw=25,200; Mn=12,100; Mw/Mn=2.1.

### EXAMPLE 30

### Statistical Copolymer Comprising 85% of 2,7-linked-(9-(2-hexyldecyl)-3,6-dimethyl-carbazole) and 15% of 1,4-linked-(2,5-bis-(n-hexyl)-benzene)

A mixture of bis(1,5-cyclooctadienyl)nickel(0) (0.97 g, 3.53 mmol), 2,2′-bipyridyl (0.55 g, 3.53 mmol) and 1,5-cyclooctadiene (0.38 g, 3.53 mmol), toluene (8 cm³) and DMF (8 cm³) was heated under an inert atmosphere to 80° C. for 30 min. A solution of 2,7-dibromo-9-(2-hexyldecyl)-3,6-dimethyl-carbazole (0.89 g, 1.547 mmol), 1,4-dibromo-2,5-bis-(n-hexyl)-benzene (0.11 g, 0.273 mmol) and 4-bromotoluene (34 mg, 0.2 mmol) in degassed toluene (8 cm³) was then added to the mixture and the reaction was then maintained at 80° C. for 24 h. The polymer was then precipitated into a solvent mixture of methanol (150 cm³), acetone (150 cm³) and concentrated hydrochloric acid (150 cm³). The polymer was then subjected to soxhlet extraction with methanol over 24 h. It was then dissolved in chloroform and the insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give the statistical polymer as a yellow powder. Soxhlet extraction with acetone over 24 h affords 0.57 g of the polymer as a deep yellow powder. (85% yield). GPC: Mw=32,500; Mn=17,100; Mw/Mn=1.9.

### EXAMPLE 31

### Statistical Copolymer Comprising 85% of 2,7-linked-(9-(2-hexyldecyl)-3,6-dimethyl-carbazole) and 15% of 3,8-linked-[1,10]phenanthroline

A mixture of bis(1,5-cyclooctadienyl)nickel(0) (1.07 g, 3.88 mmol), 2,2′-bipyridyl (0.61 g, 3.88 mmol) and 1,5-cyclooctadiene (0.42 g, 3.88 mmol), toluene (8 cm³) and DMF (8 cm³) was heated under an inert atmosphere to 80° C. for 30 min. A solution of 2,7-dibromo-9-(2-hexyldecyl)-3,6-dimethyl-carbazole (0.98 g, 1.70 mmol), 3,8-dibromo-[1,10]phenanthroline (0.10 g, 0.30 mmol) and 4-bromotoluene (37 mg, 0.22 mmol) in degassed toluene (B cm³) was then added to the mixture and the reaction was then maintained at 80° C. for 24 h. The polymer was then precipitated into a solvent mixture of methanol (150 cm³), acetone (150 cm³) and concentrated hydrochloric acid (150 cm³). It was then isolated and stirred in THF (150 cm³) with hydrazine hydrate (5 g) over 24 h. The resulting mixture was concentrated and precipitated in methanol. The polymer was then subjected to soxhlet extraction with methanol over 24 h. It was then dissolved in chloroform and the insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give the statistical polymer as a green powder. Soxhlet extraction with acetone over 24 h affords 0.64 g of the polymer as a green powder. (84% yield). GPC: Mw=26,200; Mn=13,100; Mw/Mn=2.0.

### EXAMPLE 32

### Statistical Copolymer Comprising 80% of 2,7-linked-(9-(2-hexyldecyl)-3,6-dimethyl-carbazole) and 20% of 4,4′-linked-(2,5-diphenyl-[1,3,4]oxadiazole)

A mixture of bis(1,5-cyclooctadienyl)nickel (0) (0.97 g, 3.53 mmol), 2,2′-bipyridyl (0.55 g, 3.53 mmol) and 1,5-cyclooctadiene. (0.38 g, 3.53 mmol), toluene (8 cm³) and DMF (8 cm³) was heated under an inert atmosphere to 80° C. for 30 min. A solution of 2,7-dibromo-9-(2-hexyldecyl)-3,6-dimethyl-carbazole (0.81 g, 1.40 mmol), 2,5-bis-(4-bromo-phenyl)-[1,3,4]oxadiazole (0.15 g, 0.4 mmol) and 4-bromotoluene (34 mg, 0.2 mmol) in degassed toluene (8 cm³) was then added to the mixture and the reaction was then maintained at 80° C. for 24 h. The polymer was then precipitated into a solvent mixture of methanol (150 cm³), acetone (150 cm³) and concentrated hydrochloric acid (150 cm³). The polymer was then subjected to soxhlet extraction with methanol over 24 h. It was then dissolved in chloroform and the insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more tunes. The precipitate was dried under vacuum to give the statistical polymer as a green powder. Soxhlet extraction with acetone over 44 h affords 0.57 g of the polymer as a green powder. (85% yield). GPC: Mw=24,900; Mn=13,100; Mw/Mn=1.9.

### EXAMPLE 33

### Statistical Copolymer Comprising 80% of 2,7-linked-(9-(2hexyldecyl)-3,6-dimethyl-carbazole) and 20% of 3,3′-linked-(2,5-diphenyl-[1,3,4]oxadiazole)

A mixture of bis(1,5-cyclooctadienyl)nickel(0) (0.87 g, 3.18 mmol), 2,2′-bipyridyl (0.49 g, 3.18 mmol) and 1,5-cyclooctadiene (0.34 g, 3.18 mmol), toluene (8 cm³) and DMF (8 cm³) was heated under an inert atmosphere to 80° C. for 30 min. A solution of 2,7-dibromo-9-(2-hexyldecyl)-3,6-dimethyl-carbazole (0.73 g, 1.26 mmol), 2,5-bis-(3-bromo-phenyl)-[1,3,4]oxadiazole (0.135 g, 0.36 mmol) and 4-bromotoluene (31 mg, 0.18 mmol) in degassed toluene (8 cm³) was then added to the mixture and the reaction was then maintained at 80° C. for 24 h. The polymer was then precipitated into a solvent mixture of methanol (150 cm³), acetone (150 cm³) and concentrated hydrochloric acid (150 cm³). The polymer was then subjected to soxhlet extraction with methanol over 24 h. It was then dissolved in chloroform and the insoluble materials in the chloroform solution were filtered off, after which the filtrate was concentrated in vacuo. The concentrated chloroform solution was poured into methanol under inert nitrogen atmosphere, and the precipitate was filtered off. The precipitate was re-precipitated from chloroform into methanol under inert nitrogen atmosphere two more times. The precipitate was dried under vacuum to give the statistical polymer as a yellow powder. Soxhlet extraction with acetone over 24 h affords 0.54 g of the polymer as a deep yellow powder. (80% yield). GPC: Mw=25,400; Mn=12,100; Mw/Mn=2.1.

The reader's attention is directed to all papers and documents which are filed concurrently with or previous to this specification in connection with this application and which are open to public inspection with this specification, and the contents of all such Papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1-73. (canceled)

74. A carbazole of formula I: 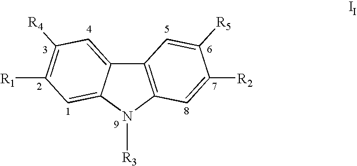
wherein R₁ and R₂ are each independently halo, B(OH)₂, B(OR₇)₂, Sn(R₈)₃, C₁₋₂₀ hydrocarbyl, or C₁₋₂₀ hydrocarbyl comprising one or more hetero atoms,
R₃ is H, halo, C₁₋₂₀ hydrocarbyl, C₁₋₂₀ hydrocarbyl comprising one or more heteroatoms, or cyano,
R₄ and R₅ are each independently H, halo, C₁₋₂₀ hydrocarbyl, C₁₋₂₀ hydrocarbyl comprising one or more heteroatoms, or cyano, and
R₇ and R₈ are each independently C₁₋₂₀ hydrocarbyl, provided that R₄ and R₅ are not both H when R₃ is n-octyl.

75. A carbazole according to claim 74, wherein R₄ and R₅ are not both hydrogen.

76. A carbazole according to claim 74, in which R₄ and R₅ are independently in each occurrence hydrogen, C₁₋₂₀ hydrocarbyl, C₁₋₂₀ hydrocarbyloxy, C₁₋₂₀ thiohydrocarbyloxy, or cyano.

77. A carbazole according to claim 74, in which R₄ and R₅ are independently in each occurrence hydrogen, C₁₋₂₀ alkyl, C₆₋₁₀ aryl or alkyl-substituted aryl, C₆₋₁₀ aryloxy or alkyl-substituted aryloxy, C₁₋₁₂ alkoxy/thioalkoxy, or cyano.

78. A carbazole according to claim 74, in which R₄ and R₅ are independently in each occurrence hydrogen, C₁₋₁₀ alkyl, phenyl, or cyano.

79. A carbazole according to claim 74, in which R₃ is hydrogen, C₁₋₂₀ hydrocarbyl, optionally substituted with one or more of C₁₋₂₀ alkoxy, C₁₋₂₀ aryloxy, C₁₋₂₀ thioalkoxy, or C₁₋₂₀ thioaryloxy groups, secondary or tertiary amine groups, hydroxy groups, carboxylic acid groups, sulphonic acid groups, cyano groups, or ester groups.

80. A carbazole according to claim 74, in which R₃ is hydrogen, C₁₋₁₂ alkyl, optionally substituted with one or more C₁₋₂ alkoxy groups, aryloxy groups, thioalkoxy groups, or thioaryloxy groups, secondary or tertiary amine groups, hydroxy groups, carboxylic acid groups, sulphonic acid groups, cyano groups, or ester groups, or C₆₋₂₀ aryl, optionally substituted with C₁₋₁₂ alkoxy groups, aryloxy groups, thioalkoxy groups, thioaryloxy groups, secondary or tertiary amine groups, hydroxy groups, carboxylic acid groups, sulphonic acid groups, cyano groups, or ester groups.

81. A carbazole according to claim 74, in which R₃ is hydrogen, C₁₋₈ alkyl, optionally substituted with one or more C₁₋₁₀ alkoxy groups, aryloxy groups, thioalkoxy groups, thioaryloxy groups, secondary or tertiary amine groups, hydroxy groups, carboxylic acid groups, sulphonic acid groups, cyano groups, or ester groups, or C₁₋₁₂ aryl, optionally substituted with C₁₋₁₀ alkoxy groups, aryloxy groups, thioalkoxy groups, thioaryloxy groups, secondary or tertiary amine groups, hydroxy groups, carboxylic acid groups, sulphonic acid groups, cyano groups, or ester groups.

82. A carbazole according to formula I that is:
2,7-dimethyl-carbazole,
2,7-dichloro-carbazole,
2,7-dibromo-carbazole,
2,7-diiodo-carbazole,
2,7-dibromo-3,6-dimethyl-carbazole,
2,7-dibromo-9-(2-ethylhexyl)-carbazole,
2,7-dibromo-9-dodecyl-carbazole,
2,7-dibromo-9-hexadecyl-carbazole,
2,7-dibromo-9-(2-hexadecyl)-carbazole,
2,7-dibromo-3,6-dimethyl-9-(2-hexyldecyl)-carbazole,
2,7-dichloro-9-dodecyl-carbazole,
2,7-dichloro-9-(2-hexadecyl)-carbazole,
2,7-bis(boronic acid)-9-hexadecyl-carbazole,
2,7-bis(4,4,5,5-tetramethyl-1,3,2-dioxaborolan-2-yl)-9-hexyldecyl-carbazole,
2,7-bis(boronic acid)-3,6-dimethyl-9-hexadecyl-carbazole,
2,7-bis(boronic acid)-9-(2-hexadecyl)-carbazole,
2,7-bis(4,4,5,5-tetramethyl-1,3,2-dioxaborolan-2-yl)-9-(2-hexadecyl)-carbazole,
2,7-bis(boronic acid)-3,6-dimethyl-9-(2-hexadecyl)-carbazole,
2,7-bis(tri-(n-butyl)stannyl)-9-(2-hexyldecyl)-carbazole, or
2,7-bis(tri-(n-methyl)stannyl)-3,6-dimethyl-9-(2-hexyldecyl)-carbazole.

83. A method for the production of a compound of formula I where R₃ is H, which comprises contacting a 4,5,4′, 5′-tetrasubstituted-biphenyl-2,2′-diamine derivative of formula IV: 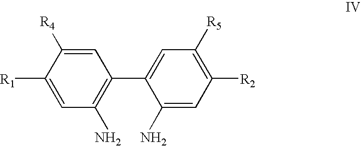
where R₁, R₂, R₄, and R₅ represent substituents as hereinbefore defined, with an arylsulphonic acid in an organic solvent at an elevated reaction temperature.

84. A method according to claim 83, in which the reaction temperature is from 180° C. to 250° C.

85. A method according to claim 83, in which the aryl sulphonic acid preferably comprises a mono-,di- or tri-substituted benzene ring wherein the one or more substituent groups is/are preferably a C₁₋₂₀ hydrocarbyl group, or a C₁₋₂₀ hydrocarbyl group containing one or more S, N, O, P or Si atoms.

86. A method according to claim 83, which comprises contacting a 4, 5, 4′, 5′-tetrasubstituted-biphenyl-2,2′—diamine derivative with an arylsulphonic acid in an organic solvent at a reflux temperature.

87. A method according to claim 85, in which the arylsulphonic acid is dodecylbenzenesulfonic acid.

88. , A method according to claim 83, in which the organic solvent is a xylene derivative.

89. A method according to claim 83, which comprises the further step of alkylating the nitrogen atom of the carbazole derivative to produce a 9-functionalised carbazole derivative.

90. A method according to claim 83, which comprises producing a 2,7-dihalo-carbazole and subjecting the 2,7-dihalo-carbazole to a metal halogen exchange reaction at low temperature and further reaction with a tri-alkoxy-borane derivative to produce a compound of formula I where R₁ and R₂ are boronic acid ester groups.

91. A method according to claim 90, which comprises the further step of hydrolysing the boronic acid ester groups to give the corresponding boronic acid groups.

92. A method according to claim 83, which comprises producing a 2,7-dihalo-carbazole and subjecting the 2,7-dihalo-carbazole to a metal halogen exchange reaction at low temperature followed by further reaction with a tri-alkyl tin chloride derivative.

93. A conjugated oligomer or polymer comprising at least 10% of the repeating unit: 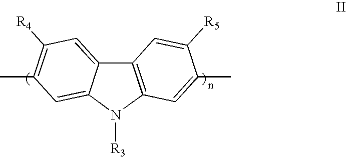
wherein R₃ is H, halo, C₁₋₂₀ hydrocarbyl, C₁₋₂₀ hydrocarbyl comprising one or more heteroatoms, or cyano, R₄ and R₅ are each independently H, halo, C₁₋₂₀ hydrocarbyl, C₁₋₂₀ hydrocarbyl comprising one or more hetero atoms, or cyano, and wherein the polymer has a degree of polymerisation n greater than 4 as measured by gel permeation chromatography.

94. An oligomer or polymer according to claim 93, wherein at least one of R₄ and R₅ is not hydrogen.

95. An oligomer or polymer according to claim 93, which is terminated at the terminal 2- and 7′-positions with hydrogen or a halogen atom.

96. An oligomer or polymer according to claim 93, wherein R₄ and R₅ are independently in each occurrence hydrogen, C₁₋₂₀ hydrocarbyl, C₁₋₂₀ hydrocarbyloxy, C₁₋₂₀ thiohydrocarbyloxy, or cyano.

97. An oligomer or polymer according to claim 93, wherein R₄ and R₅ are independently in each occurrence hydrogen, C₁₋₂₀ alkyl, C₆₋₁₀ aryl or alkyl-substituted aryl, C₆₋₁₀ aryloxy or alkyl-substituted aryloxy, C₁₋₁₂ alkoxy/thioalkoxy, or cyano.

98. An oligomer or polymer according to claim 93, wherein R₄ and R₅ are independently in each occurrence hydrogen, C₁₋₁₀ oalkyl, phenyl, or cyano.

99. An oligomer or polymer according to claim 93, in which R₃ is hydrogen, C₁₋₂₀ hydrocarbyl optionally substituted with one or more of C₁₋₂₀ alkoxy, C₁₋₂₀ aryloxy, C₁₋₂₀ thioalkoxy, or C₁₋₂₀ thioaryloxy groups, secondary or tertiary amine groups, hydroxy groups, carboxylic acid groups, sulphonic acid groups, cyano groups, or ester groups.

100. An oligomer or polymer according to claim 93, in which R₃ is hydrogen, C₁₋₁₂ alkyl, optionally substituted with one or more C₁₋₁₂ alkoxy groups, aryloxy groups, thioalkoxy groups, thioaryloxy groups, secondary or tertiary amine groups, hydroxy groups, carboxylic acid groups, sulphonic acid groups, cyano groups, ester groups, or C₆₋₂₀ aryl, optionally substituted with C₁₋₁₂ alkoxy groups, aryloxy groups, thioalkoxy groups, thioaryloxy groups, secondary or tertiary amine groups, hydroxy groups, carboxylic acid groups, sulphonic acid groups, cyano groups, or ester groups.

101. An oligomer or polymer according to claim 93, in which R₃ is hydrogen, C₁₋₈ alkyl, optionally substituted with one or more C₁₋₁₀ alkoxy groups, aryloxy groups, thioalkoxy groups, thioaryloxy groups, secondary or tertiary amine groups, hydroxy groups, carboxylic acid groups, sulphonic acid groups, cyano groups, ester groups, or C₁₋₁₂ aryl, optionally substituted with C₁₋₁₀ alkoxy groups, aryloxy groups, thioalkoxy groups, thioaryloxy groups, secondary or tertiary amine groups, hydroxy groups, carboxylic acid groups, sulphonic acid groups, cyano groups, or ester groups.

102. An oligomer or polymer according to formula II that is:
poly(9-dodecylcarbazole)-2,7-diyl.
poly(9-hexyldecylcarbazole)-2,7-diyl.
poly(9-(2-hexyldecyl)carbazole)-2,7-diyl.
poly(3,6-dibromo-9-(2-hexadecyl)-carbazole)-2,7-diyl or
poly(3,6-dimethyl-9-(2-hexyldecyl)-carbazole)-2,7-diyl.

103. A method for the production of an oligomer or polymer of formula II, which comprises subjecting a carbazole of formula I to a polycondensation reaction in the presence of a transition metal.

104. A method according to claim 103, in which the transition metal is nickel or palladium.

105. A method according to claim 103, in which the reaction is carried out in an organic solvent.

106. A method according to claim 105, in which the organic solvent is tetrahydrofuran.

107. A conjugated co-polymer of the formula: 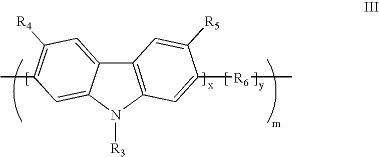
wherein R₃, R₄ and R₅ represent substituents as hereinbefore defined,
R₆ is an aryl or heteroaryl repeating unit,
0.1<x<0.9, 0.1<y<0.9, x+y=1, and
m is an integer greater than 1.

108. A co-polymer according to claim 107, wherein at least one of R₄ and R₅ is not hydrogen.

109. A co-polymer according to claim 107, which is terminated at the terminal 2- and 7′-positions with hydrogen or a halogen atom.

110. A co-polymer according to claim 107, wherein R₄ and R₅ are independently in each occurrence hydrogen, C₁₋₂₀ hydrocarbyl, C₁₋₂₀ hydrocarbyloxy, C₁₋₂₀ thiohydrocarbyloxy, or cyano.

111. A co-polymer according to claim 107, wherein R₄ and R₅ are independently in each occurrence hydrogen, C₁₋₂₀ alkyl, C₆₋₁₀ aryl or alkyl-substituted aryl, C₆₋₁₀ aryloxy or alkyl-substituted aryloxy, C₁₋₁₂ alkoxy/thioalkoxy, or cyano.

112. A co-polymer according to claim 107, wherein R₄ and R₅ are independently in each occurrence hydrogen, C₁₋₁₀ alkyl, phenyl, or cyano.

113. A co-polymer according to claim 107, in which R₃ is hydrogen, or C₁₋₂₀ hydrocarbyl, optionally substituted with one or more of C₁₋₂₀ alkoxy, C₁₋₂₀ aryloxy, C₁₋₂₀ thioalkoxy, or C₁₋₂₀ thioaryloxy groups, secondary or tertiary amine groups, hydroxy groups, carboxylic acid groups, sulphonic acid groups, cyano groups, or ester groups.

114. A co-polymer according to claim 107, in which R₃ is hydrogen, C₁₋₂₀ alkyl, optionally substituted with one or more C₁₋₁₂ alkoxy groups, aryloxy groups, thioalkoxy groups, thioaryloxy groups, secondary or tertiary amine groups, hydroxy groups, carboxylic acid groups, sulphonic acid groups, cyano groups, or ester groups, or C₆₋₂₀ aryl, optionally substituted with C₁₋₁₂ alkoxy groups, aryloxy groups, thioalkoxy groups, thioaryloxy groups, secondary or tertiary amine groups, hydroxy groups, carboxylic acid groups, sulphonic acid groups, cyano groups, or ester groups.

115. A co-polymer according to claim 107, in which R₃ is hydrogen, C₁₋₈ alkyl, optionally substituted with one or more C₁₋₁₀ alkoxy groups, aryloxy groups, thioalkoxy groups, thioaryloxy groups, secondary or tertiary amine groups, hydroxy groups, carboxylic acid groups, sulphonic acid groups, cyano groups, or ester groups, or C₁₋₁₂ aryl, optionally substituted with C₁₋₁₀ alkoxy groups, aryloxy groups, thioalkoxy groups, thioaryloxy groups, secondary or tertiary amine groups, hydroxy groups, carboxylic acid groups, sulphonic acid groups, cyano groups, or ester groups.

116. A copolymer according to claim 107, in which R₆ is a C₄₋₂₀ unsaturated ring structure containing optionally one or more heteroatoms of S, N, or O.

117. A copolymer according to claim 107, which comprises at least 50% by weight of RMUs of formula III.

118. A co-polymer according to formula III that is:
poly {(2,2′-bithiophene)-5,5′-diyl-alt-co-(9-(2-hexyldecyl)-carbazole)-2,7-diyl)},
poly{(2,5-bis(decyloxy)-benzene-1,4-diyl)-alt-co-(9-(2-hexyldecyl)-carbazole)-2,7-diyl)},
poly{(9-(2-hexyldecyl)-carbazole)-2,7-diyl)-alt-co-(naphthalene-1,4-diyl)},
a statistical copolymer comprising 85% of 2,7-linked-(9-(2-hexyldecyl)-carbazole) and 15% of 1,4-linked-naphthalene,
a statistical copolymer comprising 85% of 2,7-linked-(9-(2-hexyldecyl)-3,6-dimethyl-carbazole) and 15% of 1,4-linked-naphthalene,
a statistical copolymer comprising 85% of 2,7-linked-(9-(2-hexyldecyl)-3,6-dimethyl-carbazole) and 15% of 1,4-linked-(2,5-bis-(n-hexyl)-benzene),
a statistical copolymer comprising 85% of 2,7-linked-(9-(2-hexyldecyl)-3,6-dimethyl-carbazole) and 15% of 3,8-linked-[1,10]phenanthroline, or
a statistical copolymer comprising 80% of 2,7-linked-(9-(2-hexyldecyl)-3,6-dimethyl-carbazole) and 20% of 4,4′-linked-(2,5-diphenyl-[1,3,4]oxadiazole).

119. A polymer of claim 93, which exhibits photoluminescent emission in the range of 350 nm to 1000 nm and absorption from 200 nm to 600 nm.

120. A polymer of claim 107, which exhibits photoluminescent emission in the range of 350 nm to 1000 nm and absorption from 200 nm to 600 nm.

121. A polymer blend comprising from 1 to 99% by weight of at least one carbazole containing polymer of formula II or copolymer of formula III.

122. A method for the production of a polymer of formula II or a co-polymer of formula III, which comprises the step of electrophilic substitution of a pre-formed polymer at the 3- or 3,6-positions on carbazole repeat units.

123. A method according to claim 122, which comprises reacting a preformed polymer or co-polymer with N-bromosuccinimide or N-chlorosuccinimide.

124. A method for the production of a co-polymer of formula III, which comprises reacting a compound of formula I in a metal-catalysed coupling reaction.

125. A method according to claim 124, in which the metal is nickel or palladium.

126. A method according to claim 124, in which the reaction is carried out in an organic solvent.

127. A method according to claim 126, in which the organic solvent is tetrahydrofuran.

128. A film comprising at least 0.1 weight percent of at least one oligomer or polymer according to claim 93.

129. A film comprising at least 0.1 weight percent of at least one co-polymer according to claim 107.

130. A film according to claim 128, having a thickness of from 0.01 to 200 microns.

131. A film according to claim 129 having a thickness of from 0.01 to 200 microns.

132. A film according to claim 130, which is used as a fluorescent coating and has a thickness of from 50 to 200 microns.

133. A film according to claim 131, which is used as a fluorescent coating and has a thickness of from 50 to 200 microns.

134. A film according to claim 130, which is used as an electronic protective layer, and has a thickness of from 5 to 20 microns.

135. A film according to claim 131, which is used as an electronic protective layer, and has a thickness of from 5 to 20 microns.

136. A film according to claim 130, which is used in a polymeric light-emitting diode, and has a thickness of 0.05 to 2 microns.

137. A film according to claim 131, which is used in a polymeric light-emitting diode, and has a thickness of 0.05 to 2 microns.

138. An electronic device, especially an electroluminescent device, comprising one or more layers of the polymer film of claim 128.

139. An electronic device, especially an electroluminescent device, comprising one or more layers of the polymer film of claim 129.

140. A device according to claim 138, which is a light emitting diode, a photocell, a photo conductor or a field transistor.

141. A device according to claim 139, which is a light emitting diode, a photocell, a photo conductor or a field transistor.

142. An organic electro luminescent device, especially a light imaging diode, which comprises one or more carbazole polymers of formula II or copolymers of formula III, wherein the polymers and/or copolymers are present as single-layer films, or as multiple-layer films, whose combined thickness is in the range of 10 nm to 1000 nm, preferably in the range of 25 nm to 500 nm, most preferably in the range of 50 nm to 300 nm.

143. A device according to claim 142, which comprises a photo cell.

144. A device according to claim 143, wherein the photocell is a photovoltaic device, a solar cell, a photodiode, or a photodetector.

145. A device according to claim 142, which comprises a metal-insulator-semi conductor field effect transistor.
